SEQUENCE LISTING (70 sequences)

SEQ ID NO: 1 - DNA, 978 bp, Glycine max
  CDS (1)..(978)
cagctcacacccactttctacagggaaacctgtcccaacttgttccccattgtgttcggcgtcatcttcgatgcgtcgttcaccgaccccaggatcggagcctcgctcatgcgcctccatttccacgactgtttcgtccagggctgtgacggttccgtcttgttgaacaacaccgacaccatcgagtccgagcaggacgcgctccccaacatcaactccatccgaggcctcgatgtcgtgaacgacatcaaaaccgcagtggaaaactcctgtcccgatacggtctcctgtgcagacatcttggcgattgcagccgagatcgcatcggtcctcggaggcggtcctggctggcctgtgccgctcggacgacgggactcgttgacagcaaacaggacgctcgcaaaccagaacttgcctgcgcctttcttcaacctcacccagttgaaggcctccttcgcagtccagggcctcaacacactcgacctcgtcacactctcgggaggtcacaccttcggacgagcacgctgttcgaccttcattaaccgcctctacaacttctccaacacgggcaaccccgatcctacactcaacacaacctacttggaggtgttgcgagcacggtgtcctcagaacgcaaccggagataacctcaccaacctcgacctctcgacacccgaccagttcgacaaccgctactattcgaacttgctccagctcaacggtctcttgcagtcggaccaggagctcttctcgacacctggagcggacactatccctatcgtgaactccttctcgtcgaaccagaacaccttcttctcgaacttccgagtctccatgatcaaaatgggcaacattggagtcttgacaggtgatgagggcgaaatcaggctccagtgtaacttcgtgaacggcgactcgttcggtttggcctcggtcgcctcgaaggatgccaagcagaagctcgtcgcccagtccaaa

SEQ ID NO: 2 - protein, 326 aa, Glycine max
QLTPTFYRETCPNLFPIVFGVIFDASFTDPRIGASLMRLHFHDCFVQGCDGSVLLNNTDTIESEQDALPNINSIRGLDVVNDIKTAVENSCPDTVSCADILAIAAEIASVLGGGPGWPVPLGRRDSLTANRTLANQNLPAPFFNLTQLKASFAVQGLNTLDLVTLSGGHTFGRARCSTFINRLYNFSNTGNPDPTLNTTYLEVLRARCPQNATGDNLTNLDLSTPDQFDNRYYSNLLQLNGLLQSDQELFSTPGADTIPIVNSFSSNQNTFFSNFRVSMIKMGNIGVLTGDEGEIRLQCNFVNGDSFGLASVASKDAKQKLVAQSK

SEQ ID NO: 3 - DNA, 912 bp, Roystonea sp.
  CDS (1)..(912)
gacctccagattggattctataacacctcctgtccgaccgcagaatcgttggtccagcaggcggtggcagcagccttcgcgaacaactccggcattgcccctggcctcatccgcatgcacttccacgactgtttcgtcaggggttgtgacgcctccgtcctcttggactcgaccgccaacaacacggcagaaaaggatgcaatccccaacaacccctcgctcaggggcttcgaggtgatcaccgcagcaaagtcggcagtcgaagccgcatgtccgcagactgtgtcctgtgccgacattctcgccttcgcagcccgagactcggcgaacttggcaggcaacattacttaccaggtgccgtccggacgacgagacggcacagtgtccttggcatccgaagccaacgcgcagatcccctcccctctcttcaacgccacacagttgatcaactcgttcgcgaacaagactctcactgccgacgaaatggtcacattgtccggagcccactcgatcggcgtggcacactgttcctcgttcacgaaccgactctacaacttcaactcgggctccggcatcgacccgacactctccccttcgtacgcagcactcttgcgcaacacatgtcctgccaactccacacggttcacgcctatcaccgtgtcgttggacattatcaccccgtcggtcttggataacatgtactacaccggtgtccagctcaccttgggattgctcacctcggatcaggcactcgtgacggaagccaacttgtccgcagcggtgaaagcaaacgcaatgaacttgactgcgtgggcgtcgaagttcgcccaggccatggtgaaaatgggacagatcgaagtcctcacgggtacccagggagagatcaggaccaactgttccgtggtcaactcc

SEQ ID NO: 4 - protein, 304 aa, Roystonea sp.
DLQIGFYNTSCPTAESLVQQAVAAAFANNSGIAPGLIRMHFHDCFVRGCDASVLLDSTANNTAEKDAIPNNPSLRGFEVITAAKSAVEAACPQTVSCADILAFAARDSANLAGNITYQVPSGRRDGTVSLASEANAQIPSPLFNATQLINSFANKTLTADEMVTLSGAHSIGVAHCSSFTNRLYNFNSGSGIDPTLSPSYAALLRNTCPANSTRFTPITVSLDIITPSVLDNMYYTGVQLTLGLLTSDQALVTEANLSAAVKANAMNLTAWASKFAQAMVKMGQIEVLTGTQGEIRTNCSVVNS

SEQ ID NO: 5 - protein, 7 aa, Artificial Sequence
  Motif
HFHDCFV

SEQ ID NO: 6 - protein, 8 aa, Artificial Sequence
  Motif
GCDXSXLL

SEQ ID NO: 7 - protein, 7 aa, Artificial Sequence
  Motif
VSCXDXL

SEQ ID NO: 8 - DNA, 1059 bp, Artificial Sequence
  Artificial construct
atgggctcgatgcgcttgctcgtggtcgccctcctctgtgccttcgcgatgcatgcaggcttctcggtgtcgtatgcacagctcacacccactttctacagggaaacctgtcccaacttgttccccattgtgttcggcgtcatcttcgatgcgtcgttcaccgaccccaggatcggagcctcgctcatgcgcctccatttccacgactgtttcgtccagggctgtgacggttccgtcttgttgaacaacaccgacaccatcgagtccgagcaggacgcgctccccaacatcaactccatccgaggcctcgatgtcgtgaacgacatcaaaaccgcagtggaaaactcctgtcccgatacggtctcctgtgcagacatcttggcgattgcagccgagatcgcatcggtcctcggaggcggtcctggctggcctgtgccgctcggacgacgggactcgttgacagcaaacaggacgctcgcaaaccagaacttgcctgcgcctttcttcaacctcacccagttgaaggcctccttcgcagtccagggcctcaacacactcgacctcgtcacactctcgggaggtcacaccttcggacgagcacgctgttcgaccttcattaaccgcctctacaacttctccaacacgggcaaccccgatcctacactcaacacaacctacttggaggtgttgcgagcacggtgtcctcagaacgcaaccggagataacctcaccaacctcgacctctcgacacccgaccagttcgacaaccgctactattcgaacttgctccagctcaacggtctcttgcagtcggaccaggagctcttctcgacacctggagcggacactatccctatcgtgaactccttctcgtcgaaccagaacaccttcttctcgaacttccgagtctccatgatcaaaatgggcaacattggagtcttgacaggtgatgagggcgaaatcaggctccagtgtaacttcgtgaacggcgactcgttcggtttggcctcggtcgcctcgaaggatgccaagcagaagctcgtcgcccagtccaaatga

SEQ ID NO: 9 - protein, 352 aa, Artificial Sequence
  Synthetic Construct
MGSMRLLVVALLCAFAMHAGFSVSYAQLTPTFYRETCPNLFPIVFGVIFDASFTDPRIGASLMRLHFHDCFVQGCDGSVLLNNTDTIESEQDALPNINSIRGLDVVNDIKTAVENSCPDTVSCADILAIAAEIASVLGGGPGWPVPLGRRDSLTANRTLANQNLPAPFFNLTQLKASFAVQGLNTLDLVTLSGGHTFGRARCSTFINRLYNFSNTGNPDPTLNTTYLEVLRARCPQNATGDNLTNLDLSTPDQFDNRYYSNLLQLNGLLQSDQELFSTPGADTIPIVNSFSSNQNTFFSNFRVSMIKMGNIGVLTGDEGEIRLQCNFVNGDSFGLASVASKDAKQKLVAQSK

SEQ ID NO: 10 - DNA, 1092 bp, Artificial Sequence
  Artificial construct
atgaagttcttcaccaccatcctcagcaccgccagccttgttgctgctctccccgccgctgttgactcgaaccataccccggccgctcctgaacttgttgcccgcctaggacagctcacacccactttctacagggaaacctgtcccaacttgttccccattgtgttcggcgtcatcttcgatgcgtcgttcaccgaccccaggatcggagcctcgctcatgcgcctccatttccacgactgtttcgtccagggctgtgacggttccgtcttgttgaacaacaccgacaccatcgagtccgagcaggacgcgctccccaacatcaactccatccgaggcctcgatgtcgtgaacgacatcaaaaccgcagtggaaaactcctgtcccgatacggtctcctgtgcagacatcttggcgattgcagccgagatcgcatcggtcctcggaggcggtcctggctggcctgtgccgctcggacgacgggactcgttgacagcaaacaggacgctcgcaaaccagaacttgcctgcgcctttcttcaacctcacccagttgaaggcctccttcgcagtccagggcctcaacacactcgacctcgtcacactctcgggaggtcacaccttcggacgagcacgctgttcgaccttcattaaccgcctctacaacttctccaacacgggcaaccccgatcctacactcaacacaacctacttggaggtgttgcgagcacggtgtcctcagaacgcaaccggagataacctcaccaacctcgacctctcgacacccgaccagttcgacaaccgctactattcgaacttgctccagctcaacggtctcttgcagtcggaccaggagctcttctcgacacctggagcggacactatccctatcgtgaactccttctcgtcgaaccagaacaccttcttctcgaacttccgagtctccatgatcaaaatgggcaacattggagtcttgacaggtgatgagggcgaaatcaggctccagtgtaacttcgtgaacggcgactcgttcggtttggcctcggtcgcctcgaaggatgccaagcagaagctcgtcgcccagtccaaatga

SEQ ID NO: 11 - protein, 363 aa, Artificial Sequence
  Synthetic Construct
MKFFTTILSTASLVAALPAAVDSNHTPAAPELVARLGQLTPTFYRETCPNLFPIVFGVIFDASFTDPRIGASLMRLHFHDCFVQGCDGSVLLNNTDTIESEQDALPNINSIRGLDVVNDIKTAVENSCPDTVSCADILAIAAEIASVLGGGPGWPVPLGRRDSLTANRTLANQNLPAPFFNLTQLKASFAVQGLNTLDLVTLSGGHTFGRARCSTFINRLYNFSNTGNPDPTLNTTYLEVLRARCPQNATGDNLTNLDLSTPDQFDNRYYSNLLQLNGLLQSDQELFSTPGADTIPIVNSFSSNQNTFFSNFRVSMIKMGNIGVLTGDEGEIRLQCNFVNGDSFGLASVASKDAKQKLVAQSK

SEQ ID NO: 12 - DNA, 1056 bp, Artificial Sequence
  Artificial construct
atgaagttcttcaccaccatcctcagcaccgccagccttgttgctgctctccccgccgctgttgactccctaggacagctcacacccactttctacagggaaacctgtcccaacttgttccccattgtgttcggcgtcatcttcgatgcgtcgttcaccgaccccaggatcggagcctcgctcatgcgcctccatttccacgactgtttcgtccagggctgtgacggttccgtcttgttgaacaacaccgacaccatcgagtccgagcaggacgcgctccccaacatcaactccatccgaggcctcgatgtcgtgaacgacatcaaaaccgcagtggaaaactcctgtcccgatacggtctcctgtgcagacatcttggcgattgcagccgagatcgcatcggtcctcggaggcggtcctggctggcctgtgccgctcggacgacgggactcgttgacagcaaacaggacgctcgcaaaccagaacttgcctgcgcctttcttcaacctcacccagttgaaggcctccttcgcagtccagggcctcaacacactcgacctcgtcacactctcgggaggtcacaccttcggacgagcacgctgttcgaccttcattaaccgcctctacaacttctccaacacgggcaaccccgatcctacactcaacacaacctacttggaggtgttgcgagcacggtgtcctcagaacgcaaccggagataacctcaccaacctcgacctctcgacacccgaccagttcgacaaccgctactattcgaacttgctccagctcaacggtctcttgcagtcggaccaggagctcttctcgacacctggagcggacactatccctatcgtgaactccttctcgtcgaaccagaacaccttcttctcgaacttccgagtctccatgatcaaaatgggcaacattggagtcttgacaggtgatgagggcgaaatcaggctccagtgtaacttcgtgaacggcgactcgttcggtttggcctcggtcgcctcgaaggatgccaagcagaagctcgtcgcccagtccaaatga

SEQ ID NO: 13 - protein, 351 aa, Artificial Sequence
  Synthetic Construct
MKFFTTILSTASLVAALPAAVDSLGQLTPTFYRETCPNLFPIVFGVIFDASFTDPRIGASLMRLHFHDCFVQGCDGSVLLNNTDTIESEQDALPNINSIRGLDVVNDIKTAVENSCPDTVSCADILAIAAEIASVLGGGPGWPVPLGRRDSLTANRTLANQNLPAPFFNLTQLKASFAVQGLNTLDLVTLSGGHTFGRARCSTFINRLYNFSNTGNPDPTLNTTYLEVLRARCPQNATGDNLTNLDLSTPDQFDNRYYSNLLQLNGLLQSDQELFSTPGADTIPIVNSFSSNQNTFFSNFRVSMIKMGNIGVLTGDEGEIRLQCNFVNGDSFGLASVASKDAKQKLVAQSK

SEQ ID NO: 14 - DNA, 1086 bp, Artificial Sequence
  Artificial construct
atgagattatcgacttcgagtctcttcctttccgtgtctctgctggggaagctggccctcgggagccctttgccccaacagcagcgatatggcaaacgcctaggacagctcacacccactttctacagggaaacctgtcccaacttgttccccattgtgttcggcgtcatcttcgatgcgtcgttcaccgaccccaggatcggagcctcgctcatgcgcctccatttccacgactgtttcgtccagggctgtgacggttccgtcttgttgaacaacaccgacaccatcgagtccgagcaggacgcgctccccaacatcaactccatccgaggcctcgatgtcgtgaacgacatcaaaaccgcagtggaaaactcctgtcccgatacggtctcctgtgcagacatcttggcgattgcagccgagatcgcatcggtcctcggaggcggtcctggctggcctgtgccgctcggacgacgggactcgttgacagcaaacaggacgctcgcaaaccagaacttgcctgcgcctttcttcaacctcacccagttgaaggcctccttcgcagtccagggcctcaacacactcgacctcgtcacactctcgggaggtcacaccttcggacgagcacgctgttcgaccttcattaaccgcctctacaacttctccaacacgggcaaccccgatcctacactcaacacaacctacttggaggtgttgcgagcacggtgtcctcagaacgcaaccggagataacctcaccaacctcgacctctcgacacccgaccagttcgacaaccgctactattcgaacttgctccagctcaacggtctcttgcagtcggaccaggagctcttctcgacacctggagcggacactatccctatcgtgaactccttctcgtcgaaccagaacaccttcttctcgaacttccgagtctccatgatcaaaatgggcaacattggagtcttgacaggtgatgagggcgaaatcaggctccagtgtaacttcgtgaacggcgactcgttcggtttggcctcggtcgcctcgaaggatgccaagcagaagctcgtcgcccagtccaaatga

SEQ ID NO: 15 - protein, 361 aa, Artificial Sequence
  Synthetic Construct
MRLSTSSLFLSVSLLGKLALGSPLPQQQRYGKRLGQLTPTFYRETCPNLFPIVFGVIFDASFTDPRIGASLMRLHFHDCFVQGCDGSVLLNNTDTIESEQDALPNINSIRGLDVVNDIKTAVENSCPDTVSCADILAIAAEIASVLGGGPGWPVPLGRRDSLTANRTLANQNLPAPFFNLTQLKASFAVQGLNTLDLVTLSGGHTFGRARCSTFINRLYNFSNTGNPDPTLNTTYLEVLRARCPQNATGDNLTNLDLSTPDQFDNRYYSNLLQLNGLLQSDQELFSTPGADTIPIVNSFSSNQNTFFSNFRVSMIKMGNIGVLTGDEGEIRLQCNFVNGDSFGLASVASKDAKQKLVAQSK

SEQ ID NO: 16 - DNA, 1050 bp, Artificial Sequence
  Artificial construct
atgagattatcgacttcgagtctcttcctttccgtgtctctgctggggaagctggccctcggcctaggacagctcacacccactttctacagggaaacctgtcccaacttgttccccattgtgttcggcgtcatcttcgatgcgtcgttcaccgaccccaggatcggagcctcgctcatgcgcctccatttccacgactgtttcgtccagggctgtgacggttccgtcttgttgaacaacaccgacaccatcgagtccgagcaggacgcgctccccaacatcaactccatccgaggcctcgatgtcgtgaacgacatcaaaaccgcagtggaaaactcctgtcccgatacggtctcctgtgcagacatcttggcgattgcagccgagatcgcatcggtcctcggaggcggtcctggctggcctgtgccgctcggacgacgggactcgttgacagcaaacaggacgctcgcaaaccagaacttgcctgcgcctttcttcaacctcacccagttgaaggcctccttcgcagtccagggcctcaacacactcgacctcgtcacactctcgggaggtcacaccttcggacgagcacgctgttcgaccttcattaaccgcctctacaacttctccaacacgggcaaccccgatcctacactcaacacaacctacttggaggtgttgcgagcacggtgtcctcagaacgcaaccggagataacctcaccaacctcgacctctcgacacccgaccagttcgacaaccgctactattcgaacttgctccagctcaacggtctcttgcagtcggaccaggagctcttctcgacacctggagcggacactatccctatcgtgaactccttctcgtcgaaccagaacaccttcttctcgaacttccgagtctccatgatcaaaatgggcaacattggagtcttgacaggtgatgagggcgaaatcaggctccagtgtaacttcgtgaacggcgactcgttcggtttggcctcggtcgcctcgaaggatgccaagcagaagctcgtcgcccagtccaaatga

SEQ ID NO: 17 - protein, 349 aa, Artificial Sequence
  Synthetic Construct
MRLSTSSLFLSVSLLGKLALGLGQLTPTFYRETCPNLFPIVFGVIFDASFTDPRIGASLMRLHFHDCFVQGCDGSVLLNNTDTIESEQDALPNINSIRGLDVVNDIKTAVENSCPDTVSCADILAIAAEIASVLGGGPGWPVPLGRRDSLTANRTLANQNLPAPFFNLTQLKASFAVQGLNTLDLVTLSGGHTFGRARCSTFINRLYNFSNTGNPDPTLNTTYLEVLRARCPQNATGDNLTNLDLSTPDQFDNRYYSNLLQLNGLLQSDQELFSTPGADTIPIVNSFSSNQNTFFSNFRVSMIKMGNIGVLTGDEGEIRLQCNFVNGDSFGLASVASKDAKQKLVAQSK

SEQ ID NO: 18 - DNA, 1062 bp, Artificial Sequence
  Artificial construct
atgaagctactctctctgaccggtgtggctggtgtgcttgcgacttgcgttgcagccactcctttggtgaagcgcctaggacagctcacacccactttctacagggaaacctgtcccaacttgttccccattgtgttcggcgtcatcttcgatgcgtcgttcaccgaccccaggatcggagcctcgctcatgcgcctccatttccacgactgtttcgtccagggctgtgacggttccgtcttgttgaacaacaccgacaccatcgagtccgagcaggacgcgctccccaacatcaactccatccgaggcctcgatgtcgtgaacgacatcaaaaccgcagtggaaaactcctgtcccgatacggtctcctgtgcagacatcttggcgattgcagccgagatcgcatcggtcctcggaggcggtcctggctggcctgtgccgctcggacgacgggactcgttgacagcaaacaggacgctcgcaaaccagaacttgcctgcgcctttcttcaacctcacccagttgaaggcctccttcgcagtccagggcctcaacacactcgacctcgtcacactctcgggaggtcacaccttcggacgagcacgctgttcgaccttcattaaccgcctctacaacttctccaacacgggcaaccccgatcctacactcaacacaacctacttggaggtgttgcgagcacggtgtcctcagaacgcaaccggagataacctcaccaacctcgacctctcgacacccgaccagttcgacaaccgctactattcgaacttgctccagctcaacggtctcttgcagtcggaccaggagctcttctcgacacctggagcggacactatccctatcgtgaactccttctcgtcgaaccagaacaccttcttctcgaacttccgagtctccatgatcaaaatgggcaacattggagtcttgacaggtgatgagggcgaaatcaggctccagtgtaacttcgtgaacggcgactcgttcggtttggcctcggtcgcctcgaaggatgccaagcagaagctcgtcgcccagtccaaatga

SEQ ID NO: 19 - protein, 353 aa, Artificial Sequence
  Synthetic Construct
MKLLSLTGVAGVLATCVAATPLVKRLGQLTPTFYRETCPNLFPIVFGVIFDASFTDPRIGASLMRLHFHDCFVQGCDGSVLLNNTDTIESEQDALPNINSIRGLDVVNDIKTAVENSCPDTVSCADILAIAAEIASVLGGGPGWPVPLGRRDSLTANRTLANQNLPAPFFNLTQLKASFAVQGLNTLDLVTLSGGHTFGRARCSTFINRLYNFSNTGNPDPTLNTTYLEVLRARCPQNATGDNLTNLDLSTPDQFDNRYYSNLLQLNGLLQSDQELFSTPGADTIPIVNSFSSNQNTFFSNFRVSMIKMGNIGVLTGDEGEIRLQCNFVNGDSFGLASVASKDAKQKLVAQSK

SEQ ID NO: 20 - DNA, 1044 bp, Artificial Sequence
  Artificial construct
atgaagctactctctctgaccggtgtggctggtgtgcttgcgacttgcgttgcagccctaggacagctcacacccactttctacagggaaacctgtcccaacttgttccccattgtgttcggcgtcatcttcgatgcgtcgttcaccgaccccaggatcggagcctcgctcatgcgcctccatttccacgactgtttcgtccagggctgtgacggttccgtcttgttgaacaacaccgacaccatcgagtccgagcaggacgcgctccccaacatcaactccatccgaggcctcgatgtcgtgaacgacatcaaaaccgcagtggaaaactcctgtcccgatacggtctcctgtgcagacatcttggcgattgcagccgagatcgcatcggtcctcggaggcggtcctggctggcctgtgccgctcggacgacgggactcgttgacagcaaacaggacgctcgcaaaccagaacttgcctgcgcctttcttcaacctcacccagttgaaggcctccttcgcagtccagggcctcaacacactcgacctcgtcacactctcgggaggtcacaccttcggacgagcacgctgttcgaccttcattaaccgcctctacaacttctccaacacgggcaaccccgatcctacactcaacacaacctacttggaggtgttgcgagcacggtgtcctcagaacgcaaccggagataacctcaccaacctcgacctctcgacacccgaccagttcgacaaccgctactattcgaacttgctccagctcaacggtctcttgcagtcggaccaggagctcttctcgacacctggagcggacactatccctatcgtgaactccttctcgtcgaaccagaacaccttcttctcgaacttccgagtctccatgatcaaaatgggcaacattggagtcttgacaggtgatgagggcgaaatcaggctccagtgtaacttcgtgaacggcgactcgttcggtttggcctcggtcgcctcgaaggatgccaagcagaagctcgtcgcccagtccaaatga

SEQ ID NO: 21 - protein, 347 aa, Artificial Sequence
  Synthetic Construct
MKLLSLTGVAGVLATCVAALGQLTPTFYRETCPNLFPIVFGVIFDASFTDPRIGASLMRLHFHDCFVQGCDGSVLLNNTDTIESEQDALPNINSIRGLDVVNDIKTAVENSCPDTVSCADILAIAAEIASVLGGGPGWPVPLGRRDSLTANRTLANQNLPAPFFNLTQLKASFAVQGLNTLDLVTLSGGHTFGRARCSTFINRLYNFSNTGNPDPTLNTTYLEVLRARCPQNATGDNLTNLDLSTPDQFDNRYYSNLLQLNGLLQSDQELFSTPGADTIPIVNSFSSNQNTFFSNFRVSMIKMGNIGVLTGDEGEIRLQCNFVNGDSFGLASVASKDAKQKLVAQSK

SEQ ID NO: 22 - DNA, 1026 bp, Artificial Sequence
  Artificial construct
atgaagttcttcaccaccatcctcagcaccgccagccttgttgctgctctccccgccgctgttgactcgaaccataccccggccgctcctgaacttgttgcccgcctaggagacctccagattggattctataacacctcctgtccgaccgcagaatcgttggtccagcaggcggtggcagcagccttcgcgaacaactccggcattgcccctggcctcatccgcatgcacttccacgactgtttcgtcaggggttgtgacgcctccgtcctcttggactcgaccgccaacaacacggcagaaaaggatgcaatccccaacaacccctcgctcaggggcttcgaggtgatcaccgcagcaaagtcggcagtcgaagccgcatgtccgcagactgtgtcctgtgccgacattctcgccttcgcagcccgagactcggcgaacttggcaggcaacattacttaccaggtgccgtccggacgacgagacggcacagtgtccttggcatccgaagccaacgcgcagatcccctcccctctcttcaacgccacacagttgatcaactcgttcgcgaacaagactctcactgccgacgaaatggtcacattgtccggagcccactcgatcggcgtggcacactgttcctcgttcacgaaccgactctacaacttcaactcgggctccggcatcgacccgacactctccccttcgtacgcagcactcttgcgcaacacatgtcctgccaactccacacggttcacgcctatcaccgtgtcgttggacattatcaccccgtcggtcttggataacatgtactacaccggtgtccagctcaccttgggattgctcacctcggatcaggcactcgtgacggaagccaacttgtccgcagcggtgaaagcaaacgcaatgaacttgactgcgtgggcgtcgaagttcgcccaggccatggtgaaaatgggacagatcgaagtcctcacgggtacccagggagagatcaggaccaactgttccgtggtcaactcctga

SEQ ID NO: 23 - protein, 341 aa, Artificial Sequence
  Synthetic Construct
MKFFTTILSTASLVAALPAAVDSNHTPAAPELVARLGDLQIGFYNTSCPTAESLVQQAVAAAFANNSGIAPGLIRMHFHDCFVRGCDASVLLDSTANNTAEKDAIPNNPSLRGFEVITAAKSAVEAACPQTVSCADILAFAARDSANLAGNITYQVPSGRRDGTVSLASEANAQIPSPLFNATQLINSFANKTLTADEMVTLSGAHSIGVAHCSSFTNRLYNFNSGSGIDPTLSPSYAALLRNTCPANSTRFTPITVSLDIITPSVLDNMYYTGVQLTLGLLTSDQALVTEANLSAAVKANAMNLTAWASKFAQAMVKMGQIEVLTGTQGEIRTNCSVVNS

SEQ ID NO: 24 - DNA, 990 bp, Artificial Sequence
  Artificial construct
atgaagttcttcaccaccatcctcagcaccgccagccttgttgctgctctccccgccgctgttgactccctaggagacctccagattggattctataacacctcctgtccgaccgcagaatcgttggtccagcaggcggtggcagcagccttcgcgaacaactccggcattgcccctggcctcatccgcatgcacttccacgactgtttcgtcaggggttgtgacgcctccgtcctcttggactcgaccgccaacaacacggcagaaaaggatgcaatccccaacaacccctcgctcaggggcttcgaggtgatcaccgcagcaaagtcggcagtcgaagccgcatgtccgcagactgtgtcctgtgccgacattctcgccttcgcagcccgagactcggcgaacttggcaggcaacattacttaccaggtgccgtccggacgacgagacggcacagtgtccttggcatccgaagccaacgcgcagatcccctcccctctcttcaacgccacacagttgatcaactcgttcgcgaacaagactctcactgccgacgaaatggtcacattgtccggagcccactcgatcggcgtggcacactgttcctcgttcacgaaccgactctacaacttcaactcgggctccggcatcgacccgacactctccccttcgtacgcagcactcttgcgcaacacatgtcctgccaactccacacggttcacgcctatcaccgtgtcgttggacattatcaccccgtcggtcttggataacatgtactacaccggtgtccagctcaccttgggattgctcacctcggatcaggcactcgtgacggaagccaacttgtccgcagcggtgaaagcaaacgcaatgaacttgactgcgtgggcgtcgaagttcgcccaggccatggtgaaaatgggacagatcgaagtcctcacgggtacccagggagagatcaggaccaactgttccgtggtcaactcctga

SEQ ID NO: 25 - protein, 329 aa, Artificial Sequence
  Synthetic Construct
MKFFTTILSTASLVAALPAAVDSLGDLQIGFYNTSCPTAESLVQQAVAAAFANNSGIAPGLIRMHFHDCFVRGCDASVLLDSTANNTAEKDAIPNNPSLRGFEVITAAKSAVEAACPQTVSCADILAFAARDSANLAGNITYQVPSGRRDGTVSLASEANAQIPSPLFNATQLINSFANKTLTADEMVTLSGAHSIGVAHCSSFTNRLYNFNSGSGIDPTLSPSYAALLRNTCPANSTRFTPITVSLDIITPSVLDNMYYTGVQLTLGLLTSDQALVTEANLSAAVKANAMNLTAWASKFAQAMVKMGQIEVLTGTQGEIRTNCSVVNS

SEQ ID NO: 26 - DNA, 1020 bp, Artificial Sequence
  Artificial construct
atgagattatcgacttcgagtctcttcctttccgtgtctctgctggggaagctggccctcgggagccctttgccccaacagcagcgatatggcaaacgcctaggagacctccagattggattctataacacctcctgtccgaccgcagaatcgttggtccagcaggcggtggcagcagccttcgcgaacaactccggcattgcccctggcctcatccgcatgcacttccacgactgtttcgtcaggggttgtgacgcctccgtcctcttggactcgaccgccaacaacacggcagaaaaggatgcaatccccaacaacccctcgctcaggggcttcgaggtgatcaccgcagcaaagtcggcagtcgaagccgcatgtccgcagactgtgtcctgtgccgacattctcgccttcgcagcccgagactcggcgaacttggcaggcaacattacttaccaggtgccgtccggacgacgagacggcacagtgtccttggcatccgaagccaacgcgcagatcccctcccctctcttcaacgccacacagttgatcaactcgttcgcgaacaagactctcactgccgacgaaatggtcacattgtccggagcccactcgatcggcgtggcacactgttcctcgttcacgaaccgactctacaacttcaactcgggctccggcatcgacccgacactctccccttcgtacgcagcactcttgcgcaacacatgtcctgccaactccacacggttcacgcctatcaccgtgtcgttggacattatcaccccgtcggtcttggataacatgtactacaccggtgtccagctcaccttgggattgctcacctcggatcaggcactcgtgacggaagccaacttgtccgcagcggtgaaagcaaacgcaatgaacttgactgcgtgggcgtcgaagttcgcccaggccatggtgaaaatgggacagatcgaagtcctcacgggtacccagggagagatcaggaccaactgttccgtggtcaactcctga

SEQ ID NO: 27 - protein, 339 aa, Artificial Sequence
  Synthetic Construct
MRLSTSSLFLSVSLLGKLALGSPLPQQQRYGKRLGDLQIGFYNTSCPTAESLVQQAVAAAFANNSGIAPGLIRMHFHDCFVRGCDASVLLDSTANNTAEKDAIPNNPSLRGFEVITAAKSAVEAACPQTVSCADILAFAARDSANLAGNITYQVPSGRRDGTVSLASEANAQIPSPLFNATQLINSFANKTLTADEMVTLSGAHSIGVAHCSSFTNRLYNFNSGSGIDPTLSPSYAALLRNTCPANSTRFTPITVSLDIITPSVLDNMYYTGVQLTLGLLTSDQALVTEANLSAAVKANAMNLTAWASKFAQAMVKMGQIEVLTGTQGEIRTNCSVVNS

SEQ ID NO: 28 - DNA, 984 bp, Artificial Sequence
  Artificial construct
atgagattatcgacttcgagtctcttcctttccgtgtctctgctggggaagctggccctcggcctaggagacctccagattggattctataacacctcctgtccgaccgcagaatcgttggtccagcaggcggtggcagcagccttcgcgaacaactccggcattgcccctggcctcatccgcatgcacttccacgactgtttcgtcaggggttgtgacgcctccgtcctcttggactcgaccgccaacaacacggcagaaaaggatgcaatccccaacaacccctcgctcaggggcttcgaggtgatcaccgcagcaaagtcggcagtcgaagccgcatgtccgcagactgtgtcctgtgccgacattctcgccttcgcagcccgagactcggcgaacttggcaggcaacattacttaccaggtgccgtccggacgacgagacggcacagtgtccttggcatccgaagccaacgcgcagatcccctcccctctcttcaacgccacacagttgatcaactcgttcgcgaacaagactctcactgccgacgaaatggtcacattgtccggagcccactcgatcggcgtggcacactgttcctcgttcacgaaccgactctacaacttcaactcgggctccggcatcgacccgacactctccccttcgtacgcagcactcttgcgcaacacatgtcctgccaactccacacggttcacgcctatcaccgtgtcgttggacattatcaccccgtcggtcttggataacatgtactacaccggtgtccagctcaccttgggattgctcacctcggatcaggcactcgtgacggaagccaacttgtccgcagcggtgaaagcaaacgcaatgaacttgactgcgtgggcgtcgaagttcgcccaggccatggtgaaaatgggacagatcgaagtcctcacgggtacccagggagagatcaggaccaactgttccgtggtcaactcctga

SEQ ID NO: 29 - protein, 327 aa, Artificial Sequence
  Synthetic Construct
MRLSTSSLFLSVSLLGKLALGLGDLQIGFYNTSCPTAESLVQQAVAAAFANNSGIAPGLIRMHFHDCFVRGCDASVLLDSTANNTAEKDAIPNNPSLRGFEVITAAKSAVEAACPQTVSCADILAFAARDSANLAGNITYQVPSGRRDGTVSLASEANAQIPSPLFNATQLINSFANKTLTADEMVTLSGAHSIGVAHCSSFTNRLYNFNSGSGIDPTLSPSYAALLRNTCPANSTRFTPITVSLDIITPSVLDNMYYTGVQLTLGLLTSDQALVTEANLSAAVKANAMNLTAWASKFAQAMVKMGQIEVLTGTQGEIRTNCSVVNS

SEQ ID NO: 30 - DNA, 1026 bp, Artificial Sequence; identical to SEQ ID NO: 22
  Artificial construct

SEQ ID NO: 31 - protein, 341 aa, Artificial Sequence; identical to SEQ ID NO: 23
  Synthetic Construct

SEQ ID NO: 32 - DNA, 978 bp, Artificial Sequence
  Artificial construct
atgaagctactctctctgaccggtgtggctggtgtgcttgcgacttgcgttgcagccctaggagacctccagattggattctataacacctcctgtccgaccgcagaatcgttggtccagcaggcggtggcagcagccttcgcgaacaactccggcattgcccctggcctcatccgcatgcacttccacgactgtttcgtcaggggttgtgacgcctccgtcctcttggactcgaccgccaacaacacggcagaaaaggatgcaatccccaacaacccctcgctcaggggcttcgaggtgatcaccgcagcaaagtcggcagtcgaagccgcatgtccgcagactgtgtcctgtgccgacattctcgccttcgcagcccgagactcggcgaacttggcaggcaacattacttaccaggtgccgtccggacgacgagacggcacagtgtccttggcatccgaagccaacgcgcagatcccctcccctctcttcaacgccacacagttgatcaactcgttcgcgaacaagactctcactgccgacgaaatggtcacattgtccggagcccactcgatcggcgtggcacactgttcctcgttcacgaaccgactctacaacttcaactcgggctccggcatcgacccgacactctccccttcgtacgcagcactcttgcgcaacacatgtcctgccaactccacacggttcacgcctatcaccgtgtcgttggacattatcaccccgtcggtcttggataacatgtactacaccggtgtccagctcaccttgggattgctcacctcggatcaggcactcgtgacggaagccaacttgtccgcagcggtgaaagcaaacgcaatgaacttgactgcgtgggcgtcgaagttcgcccaggccatggtgaaaatgggacagatcgaagtcctcacgggtacccagggagagatcaggaccaactgttccgtggtcaactcctga

SEQ ID NO: 33 - protein, 325 aa, Artificial Sequence
  Synthetic Construct
MKLLSLTGVAGVLATCVAALGDLQIGFYNTSCPTAESLVQQAVAAAFANNSGIAPGLIRMHFHDCFVRGCDASVLLDSTANNTAEKDAIPNNPSLRGFEVITAAKSAVEAACPQTVSCADILAFAARDSANLAGNITYQVPSGRRDGTVSLASEANAQIPSPLFNATQLINSFANKTLTADEMVTLSGAHSIGVAHCSSFTNRLYNFNSGSGIDPTLSPSYAALLRNTCPANSTRFTPITVSLDIITPSVLDNMYYTGVQLTLGLLTSDQALVTEANLSAAVKANAMNLTAWASKFAQAMVKMGQIEVLTGTQGEIRTNCSVVNS

SEQ ID NO: 34 - DNA, 993 bp, Artificial Sequence
  Artificial construct
atgggctccatgcgattgctcgtcgtcgcactcttgtgtgccttcgccatgcacgcaggtttctcggtgtcgtatgccgacctccagattggattctataacacctcctgtccgaccgcagaatcgttggtccagcaggcggtggcagcagccttcgcgaacaactccggcattgcccctggcctcatccgcatgcacttccacgactgtttcgtcaggggttgtgacgcctccgtcctcttggactcgaccgccaacaacacggcagaaaaggatgcaatccccaacaacccctcgctcaggggcttcgaggtgatcaccgcagcaaagtcggcagtcgaagccgcatgtccgcagactgtgtcctgtgccgacattctcgccttcgcagcccgagactcggcgaacttggcaggcaacattacttaccaggtgccgtccggacgacgagacggcacagtgtccttggcatccgaagccaacgcgcagatcccctcccctctcttcaacgccacacagttgatcaactcgttcgcgaacaagactctcactgccgacgaaatggtcacattgtccggagcccactcgatcggcgtggcacactgttcctcgttcacgaaccgactctacaacttcaactcgggctccggcatcgacccgacactctccccttcgtacgcagcactcttgcgcaacacatgtcctgccaactccacacggttcacgcctatcaccgtgtcgttggacattatcaccccgtcggtcttggataacatgtactacaccggtgtccagctcaccttgggattgctcacctcggatcaggcactcgtgacggaagccaacttgtccgcagcggtgaaagcaaacgcaatgaacttgactgcgtgggcgtcgaagttcgcccaggccatggtgaaaatgggacagatcgaagtcctcacgggtacccagggagagatcaggaccaactgttccgtggtcaactcctga

SEQ ID NO: 35 - protein, 330 aa, Artificial Sequence
  Synthetic Construct
MGSMRLLVVALLCAFAMHAGFSVSYADLQIGFYNTSCPTAESLVQQAVAAAFANNSGIAPGLIRMHFHDCFVRGCDASVLLDSTANNTAEKDAIPNNPSLRGFEVITAAKSAVEAACPQTVSCADILAFAARDSANLAGNITYQVPSGRRDGTVSLASEANAQIPSPLFNATQLINSFANKTLTADEMVTLSGAHSIGVAHCSSFTNRLYNFNSGSGIDPTLSPSYAALLRNTCPANSTRFTPITVSLDIITPSVLDNMYYTGVQLTLGLLTSDQALVTEANLSAAVKANAMNLTAWASKFAQAMVKMGQIEVLTGTQGEIRTNCSVVNS

SEQ ID NO: 36 - DNA, 31 bp, Artificial Sequence
  Primer 1
tcctgacctaggacagctcacacccactttc

SEQ ID NO: 37 - DNA, 31 bp, Artificial Sequence
  Primer 2
acaggtcttaagtcatttggactgggcgacg

SEQ ID NO: 38 - DNA, 37 bp, Artificial Sequence
  Primer 3
tgcccgcctaggagacctccagattggattctataac

SEQ ID NO: 39 - DNA, 37 bp, Artificial Sequence
  Primer 4
atcatacttaagttatcaggagttgaccacggaacag

SEQ ID NO: 40 - DNA, 32 bp, Artificial Sequence
  Primer 5
taatcctaggtcagctcacacctaccttctac

SEQ ID NO: 41 - DNA, 22 bp, Artificial Sequence
  Primer 6
ggtacccttaagtcaaatcgac

SEQ ID NO: 42 - DNA, 35 bp, Artificial Sequence
  Primer 7
taatcctaggtgccggtctcaaagtgggattctac

SEQ ID NO: 43 - DNA, 28 bp, Artificial Sequence
  Primer 8
attacttaagtcagttggttgccacgtg

SEQ ID NO: 44 - DNA, 1077 bp, Populus sp.
  CDS (7)..(1068)
ggatccatggaaagggtcttctccttcaaaatgatgatcgacaaggccctccacccgttggtcgcatcgctcttcttcgtgatctggttcggtggctcgctcccctacgcatacgcccagctcacacctaccttctacgacggcacctgtcccaacgtgtcgaccatcattcgcggtgtgctcgcacaggcgttgcagaccgatccgcgaattggcgcatcgttgattcggttgcacttccatgactgtttcgtcgatggttgtgacggctcgatcctcctcgataacacggacacaatcgagtccgaaaaagaggcagcacccaacaacaactcggcaaggggcttcgatgtcgtcgataacatgaaagccgcagtcgagaacgcctgtccgggtatcgtctcgtgtgcggacatcctcgccattgcagcggaggaatcggtgcgcttggcaggcggtccctcctggaccgtcccgctcggacgacgggattccttgatcgcaaaccgatcgggagcaaactcctcgattcctgcaccctccgaatccctcgcagtgctcaaatcgaagttcgcagccgtcggcttgaacacgtcgtccgacttggtcgcgttgtcgggagcacatacgttcggtagggcacagtgtttgaacttcatttcgaggctctacaacttctcgggctcgggcaaccccgaccccacattgaacactacttacctcgcagcgctccagcagttgtgtccgcagggaggtaaccgatccgtgttgaccaacctcgaccgaacaacacccgacaccttcgacggcaactacttctccaacctccagaccaacgaaggcttgctccagtccgatcaggagttgttctccacaacaggagccgacacgatcgcgattgtcaacaacttctcctccaaccagacagccttcttcgagtccttcgtcgtctcgatgatccggatgggaaacatctcgcccttgaccggcaccgatggtgaaattcggttgaactgtcgaatcgtgaacaactccaccggctccaacgcgctcctcgtctcgtcgatttgacttaag

SEQ ID NO: 45 - protein, 354 aa, Populus sp.
MERVFSFKMMIDKALHPLVASLFFVIWFGGSLPYAYAQLTPTFYDGTCPNVSTIIRGVLAQALQTDPRIGASLIRLHFHDCFVDGCDGSILLDNTDTIESEKEAAPNNNSARGFDVVDNMKAAVENACPGIVSCADILAIAAEESVRLAGGPSWTVPLGRRDSLIANRSGANSSIPAPSESLAVLKSKFAAVGLNTSSDLVALSGAHTFGRAQCLNFISRLYNFSGSGNPDPTLNTTYLAALQQLCPQGGNRSVLTNLDRTTPDTFDGNYFSNLQTNEGLLQSDQELFSTTGADTIAIVNNFSSNQTAFFESFVVSMIRMGNISPLTGTDGEIRLNCRIVNNSTGSNALLVSSI

SEQ ID NO: 46 - DNA, 1065 bp, Artificial Sequence
  Artificial construct
atgaagttcttcaccaccatcctcagcaccgccagccttgttgctgctctccccgccgctgttgactcgaaccataccccggccgctcctgaacttgttgcccgcctaggtcagctcacacctaccttctacgacggcacctgtcccaacgtgtcgaccatcattcgcggtgtgctcgcacaggcgttgcagaccgatccgcgaattggcgcatcgttgattcggttgcacttccatgactgtttcgtcgatggttgtgacggctcgatcctcctcgataacacggacacaatcgagtccgaaaaagaggcagcacccaacaacaactcggcaaggggcttcgatgtcgtcgataacatgaaagccgcagtcgagaacgcctgtccgggtatcgtctcgtgtgcggacatcctcgccattgcagcggaggaatcggtgcgcttggcaggcggtccctcctggaccgtcccgctcggacgacgggattccttgatcgcaaaccgatcgggagcaaactcctcgattcctgcaccctccgaatccctcgcagtgctcaaatcgaagttcgcagccgtcggcttgaacacgtcgtccgacttggtcgcgttgtcgggagcacatacgttcggtagggcacagtgtttgaacttcatttcgaggctctacaacttctcgggctcgggcaaccccgaccccacattgaacactacttacctcgcagcgctccagcagttgtgtccgcagggaggtaaccgatccgtgttgaccaacctcgaccgaacaacacccgacaccttcgacggcaactacttctccaacctccagaccaacgaaggcttgctccagtccgatcaggagttgttctccacaacaggagccgacacgatcgcgattgtcaacaacttctcctccaaccagacagccttcttcgagtccttcgtcgtctcgatgatccggatgggaaacatctcgcccttgaccggcaccgatggtgaaattcggttgaactgtcgaatcgtgaacaactccaccggctccaacgcgctcctcgtctcgtcgatttga

SEQ ID NO: 47 - protein, 354 aa, Artificial Sequence
  Synthetic Construct
MKFFTTILSTASLVAALPAAVDSNHTPAAPELVARLGQLTPTFYDGTCPNVSTIIRGVLAQALQTDPRIGASLIRLHFHDCFVDGCDGSILLDNTDTIESEKEAAPNNNSARGFDVVDNMKAAVENACPGIVSCADILAIAAEESVRLAGGPSWTVPLGRRDSLIANRSGANSSIPAPSESLAVLKSKFAAVGLNTSSDLVALSGAHTFGRAQCLNFISRLYNFSGSGNPDPTLNTTYLAALQQLCPQGGNRSVLTNLDRTTPDTFDGNYFSNLQTNEGLLQSDQELFSTTGADTIAIVNNFSSNQTAFFESFVVSMIRMGNISPLTGTDGEIRLNCRIVNNSTGSNALLVSSI

SEQ ID NO: 48 - DNA, 1029 bp, Artificial Sequence
  Artificial construct
atgaagttcttcaccaccatcctcagcaccgccagccttgttgctgctctccccgccgctgttgactccctaggtcagctcacacctaccttctacgacggcacctgtcccaacgtgtcgaccatcattcgcggtgtgctcgcacaggcgttgcagaccgatccgcgaattggcgcatcgttgattcggttgcacttccatgactgtttcgtcgatggttgtgacggctcgatcctcctcgataacacggacacaatcgagtccgaaaaagaggcagcacccaacaacaactcggcaaggggcttcgatgtcgtcgataacatgaaagccgcagtcgagaacgcctgtccgggtatcgtctcgtgtgcggacatcctcgccattgcagcggaggaatcggtgcgcttggcaggcggtccctcctggaccgtcccgctcggacgacgggattccttgatcgcaaaccgatcgggagcaaactcctcgattcctgcaccctccgaatccctcgcagtgctcaaatcgaagttcgcagccgtcggcttgaacacgtcgtccgacttggtcgcgttgtcgggagcacatacgttcggtagggcacagtgtttgaacttcatttcgaggctctacaacttctcgggctcgggcaaccccgaccccacattgaacactacttacctcgcagcgctccagcagttgtgtccgcagggaggtaaccgatccgtgttgaccaacctcgaccgaacaacacccgacaccttcgacggcaactacttctccaacctccagaccaacgaaggcttgctccagtccgatcaggagttgttctccacaacaggagccgacacgatcgcgattgtcaacaacttctcctccaaccagacagccttcttcgagtccttcgtcgtctcgatgatccggatgggaaacatctcgcccttgaccggcaccgatggtgaaattcggttgaactgtcgaatcgtgaacaactccaccggctccaacgcgctcctcgtctcgtcgatttga

SEQ ID NO: 49 - protein, 342 aa, Artificial Sequence
  Synthetic Construct
MKFFTTILSTASLVAALPAAVDSLGQLTPTFYDGTCPNVSTIIRGVLAQALQTDPRIGASLIRLHFHDCFVDGCDGSILLDNTDTIESEKEAAPNNNSARGFDVVDNMKAAVENACPGIVSCADILAIAAEESVRLAGGPSWTVPLGRRDSLIANRSGANSSIPAPSESLAVLKSKFAAVGLNTSSDLVALSGAHTFGRAQCLNFISRLYNFSGSGNPDPTLNTTYLAALQQLCPQGGNRSVLTNLDRTTPDTFDGNYFSNLQTNEGLLQSDQELFSTTGADTIAIVNNFSSNQTAFFESFVVSMIRMGNISPLTGTDGEIRLNCRIVNNSTGSNALLVSSI

SEQ ID NO: 50 - DNA, 1059 bp, Artificial Sequence
  Artificial construct
atgagattatcgacttcgagtctcttcctttccgtgtctctgctggggaagctggccctcgggagccctttgccccaacagcagcgatatggcaaacgcctaggtcagctcacacctaccttctacgacggcacctgtcccaacgtgtcgaccatcattcgcggtgtgctcgcacaggcgttgcagaccgatccgcgaattggcgcatcgttgattcggttgcacttccatgactgtttcgtcgatggttgtgacggctcgatcctcctcgataacacggacacaatcgagtccgaaaaagaggcagcacccaacaacaactcggcaaggggcttcgatgtcgtcgataacatgaaagccgcagtcgagaacgcctgtccgggtatcgtctcgtgtgcggacatcctcgccattgcagcggaggaatcggtgcgcttggcaggcggtccctcctggaccgtcccgctcggacgacgggattccttgatcgcaaaccgatcgggagcaaactcctcgattcctgcaccctccgaatccctcgcagtgctcaaatcgaagttcgcagccgtcggcttgaacacgtcgtccgacttggtcgcgttgtcgggagcacatacgttcggtagggcacagtgtttgaacttcatttcgaggctctacaacttctcgggctcgggcaaccccgaccccacattgaacactacttacctcgcagcgctccagcagttgtgtccgcagggaggtaaccgatccgtgttgaccaacctcgaccgaacaacacccgacaccttcgacggcaactacttctccaacctccagaccaacgaaggcttgctccagtccgatcaggagttgttctccacaacaggagccgacacgatcgcgattgtcaacaacttctcctccaaccagacagccttcttcgagtccttcgtcgtctcgatgatccggatgggaaacatctcgcccttgaccggcaccgatggtgaaattcggttgaactgtcgaatcgtgaacaactccaccggctccaacgcgctcctcgtctcgtcgatttga

SEQ ID NO: 51 - protein, 352 aa, Artificial Sequence
  Synthetic Construct
MRLSTSSLFLSVSLLGKLALGSPLPQQQRYGKRLGQLTPTFYDGTCPNVSTIIRGVLAQALQTDPRIGASLIRLHFHDCFVDGCDGSILLDNTDTIESEKEAAPNNNSARGFDVVDNMKAAVENACPGIVSCADILAIAAEESVRLAGGPSWTVPLGRRDSLIANRSGANSSIPAPSESLAVLKSKFAAVGLNTSSDLVALSGAHTFGRAQCLNFISRLYNFSGSGNPDPTLNTTYLAALQQLCPQGGNRSVLTNLDRTTPDTFDGNYFSNLQTNEGLLQSDQELFSTTGADTIAIVNNFSSNQTAFFESFVVSMIRMGNISPLTGTDGEIRLNCRIVNNSTGSNALLVSSI

SEQ ID NO: 52 - DNA, 1035 bp, Artificial Sequence
  Artificial construct
atgaagctactctctctgaccggtgtggctggtgtgcttgcgacttgcgttgcagccactcctttggtgaagcgcctaggtcagctcacacctaccttctacgacggcacctgtcccaacgtgtcgaccatcattcgcggtgtgctcgcacaggcgttgcagaccgatccgcgaattggcgcatcgttgattcggttgcacttccatgactgtttcgtcgatggttgtgacggctcgatcctcctcgataacacggacacaatcgagtccgaaaaagaggcagcacccaacaacaactcggcaaggggcttcgatgtcgtcgataacatgaaagccgcagtcgagaacgcctgtccgggtatcgtctcgtgtgcggacatcctcgccattgcagcggaggaatcggtgcgcttggcaggcggtccctcctggaccgtcccgctcggacgacgggattccttgatcgcaaaccgatcgggagcaaactcctcgattcctgcaccctccgaatccctcgcagtgctcaaatcgaagttcgcagccgtcggcttgaacacgtcgtccgacttggtcgcgttgtcgggagcacatacgttcggtagggcacagtgtttgaacttcatttcgaggctctacaacttctcgggctcgggcaaccccgaccccacattgaacactacttacctcgcagcgctccagcagttgtgtccgcagggaggtaaccgatccgtgttgaccaacctcgaccgaacaacacccgacaccttcgacggcaactacttctccaacctccagaccaacgaaggcttgctccagtccgatcaggagttgttctccacaacaggagccgacacgatcgcgattgtcaacaacttctcctccaaccagacagccttcttcgagtccttcgtcgtctcgatgatccggatgggaaacatctcgcccttgaccggcaccgatggtgaaattcggttgaactgtcgaatcgtgaacaactccaccggctccaacgcgctcctcgtctcgtcgatttga

SEQ ID NO: 53 - protein, 344 aa, Artificial Sequence
  Synthetic Construct
MKLLSLTGVAGVLATCVAATPLVKRLGQLTPTFYDGTCPNVSTIIRGVLAQALQTDPRIGASLIRLHFHDCFVDGCDGSILLDNTDTIESEKEAAPNNNSARGFDVVDNMKAAVENACPGIVSCADILAIAAEESVRLAGGPSWTVPLGRRDSLIANRSGANSSIPAPSESLAVLKSKFAAVGLNTSSDLVALSGAHTFGRAQCLNFISRLYNFSGSGNPDPTLNTTYLAALQQLCPQGGNRSVLTNLDRTTPDTFDGNYFSNLQTNEGLLQSDQELFSTTGADTIAIVNNFSSNQTAFFESFVVSMIRMGNISPLTGTDGEIRLNCRIVNNSTGSNALLVSSI

SEQ ID NO: 54 - DNA, 1101 bp, Zea mays
  CDS (7)..(1092)
ggatccatgggaggcgtgcgctcgtacttcttcatcattgcagcagccgtcgtggcggtcgtcctcgccttgttgcctgcaggcgcaacgggagccggtctcaaagtgggattctactcgaaaacgtgtccctcggcagagtcgctcgtccagcaggccgtcgcagcggcattcaagaacaactcgggcatcgcagccggtttgatccggttgcacttccacgactgtttcgtgcgaggatgtgacggctccgtcttgattgactcgactgccaacaacacagccgaaaaggatgcagtgcccaacaacccgtccttgcgtggtttcgaggtgatcgacgcagccaagaaagcggtggaagcacgctgtcccaagacagtctcctgtgccgacatcttggcattcgcagcacgagactccatcgcactcgcaggcaacaacttgacctacaaagtgcctgcgggacgacgggatggtcgcgtgtcgagggatacggacgcaaactcgaacctcccttcccctctctccacagcagcggagctcgtcggcaacttcacacgcaagaacctcactgccgaggatatggtcgtcctctccggtgcacatactgtcggacggtcccactgttcgtccttcaccaaccgcttgtatggattctcgaacgcatcggacgtggaccccaccatttcgtcggcctacgcactcttgctccgagccatttgtccttccaacacctcccagttcttccccaacacaactacggatatggacttgattacccctgcgctcttggataaccgatactacgtgggactcgccaacaacctcggtctcttcacatccgatcaggcgttgctcaccaacgcaaccctcaagaagtccgtcgatgccttcgtcaagtccgagtcggcatggaaaaccaagttcgccaagtcgatggtcaaaatgggcaacatcgatgtgttgaccggaacgaaaggtgagatcaggctcaactgtcgggtcatcaactccggctcctcgtcctcgggcttgttccagctccacacagccacagcatcggacgaagaattcgcccacgtggcaaccaactgacttaag

SEQ ID NO: 55 - protein, 362 aa, Zea mays
MGGVRSYFFIIAAAVVAVVLALLPAGATGAGLKVGFYSKTCPSAESLVQQAVAAAFKNNSGIAAGLIRLHFHDCFVRGCDGSVLIDSTANNTAEKDAVPNNPSLRGFEVIDAAKKAVEARCPKTVSCADILAFAARDSIALAGNNLTYKVPAGRRDGRVSRDTDANSNLPSPLSTAAELVGNFTRKNLTAEDMVVLSGAHTVGRSHCSSFTNRLYGFSNASDVDPTISSAYALLLRAICPSNTSQFFPNTTTDMDLITPALLDNRYYVGLANNLGLFTSDQALLTNATLKKSVDAFVKSESAWKTKFAKSMVKMGNIDVLTGTKGEIRLNCRVINSGSSSSGLFQLHTATASDEEFAHVATN

SEQ ID NO: 56 - DNA, 1113 bp, Artificial Sequence
  Artificial construct
atgaagttcttcaccaccatcctcagcaccgccagccttgttgctgctctccccgccgctgttgactcgaaccataccccggccgctcctgaacttgttgcccgcctaggtgccggtctcaaagtgggattctactcgaaaacgtgtccctcggcagagtcgctcgtccagcaggccgtcgcagcggcattcaagaacaactcgggcatcgcagccggtttgatccggttgcacttccacgactgtttcgtgcgaggatgtgacggctccgtcttgattgactcgactgccaacaacacagccgaaaaggatgcagtgcccaacaacccgtccttgcgtggtttcgaggtgatcgacgcagccaagaaagcggtggaagcacgctgtcccaagacagtctcctgtgccgacatcttggcattcgcagcacgagactccatcgcactcgcaggcaacaacttgacctacaaagtgcctgcgggacgacgggatggtcgcgtgtcgagggatacggacgcaaactcgaacctcccttcccctctctccacagcagcggagctcgtcggcaacttcacacgcaagaacctcactgccgaggatatggtcgtcctctccggtgcacatactgtcggacggtcccactgttcgtccttcaccaaccgcttgtatggattctcgaacgcatcggacgtggaccccaccatttcgtcggcctacgcactcttgctccgagccatttgtccttccaacacctcccagttcttccccaacacaactacggatatggacttgattacccctgcgctcttggataaccgatactacgtgggactcgccaacaacctcggtctcttcacatccgatcaggcgttgctcaccaacgcaaccctcaagaagtccgtcgatgccttcgtcaagtccgagtcggcatggaaaaccaagttcgccaagtcgatggtcaaaatgggcaacatcgatgtgttgaccggaacgaaaggtgagatcaggctcaactgtcgggtcatcaactccggctcctcgtcctcgggcttgttccagctccacacagccacagcatcggacgaagaattcgcccacgtggcaaccaactga

SEQ ID NO: 57 - protein, 370 aa, Artificial Sequence
  Synthetic Construct
MKFFTTILSTASLVAALPAAVDSNHTPAAPELVARLGAGLKVGFYSKTCPSAESLVQQAVAAAFKNNSGIAAGLIRLHFHDCFVRGCDGSVLIDSTANNTAEKDAVPNNPSLRGFEVIDAAKKAVEARCPKTVSCADILAFAARDSIALAGNNLTYKVPAGRRDGRVSRDTDANSNLPSPLSTAAELVGNFTRKNLTAEDMVVLSGAHTVGRSHCSSFTNRLYGFSNASDVDPTISSAYALLLRAICPSNTSQFFPNTTTDMDLITPALLDNRYYVGLANNLGLFTSDQALLTNATLKKSVDAFVKSESAWKTKFAKSMVKMGNIDVLTGTKGEIRLNCRVINSGSSSSGLFQLHTATASDEEFAHVATN

SEQ ID NO: 58 - DNA, 1077 bp, Artificial Sequence
  Artificial construct
atgaagttcttcaccaccatcctcagcaccgccagccttgttgctgctctccccgccgctgttgactccctaggtgccggtctcaaagtgggattctactcgaaaacgtgtccctcggcagagtcgctcgtccagcaggccgtcgcagcggcattcaagaacaactcgggcatcgcagccggtttgatccggttgcacttccacgactgtttcgtgcgaggatgtgacggctccgtcttgattgactcgactgccaacaacacagccgaaaaggatgcagtgcccaacaacccgtccttgcgtggtttcgaggtgatcgacgcagccaagaaagcggtggaagcacgctgtcccaagacagtctcctgtgccgacatcttggcattcgcagcacgagactccatcgcactcgcaggcaacaacttgacctacaaagtgcctgcgggacgacgggatggtcgcgtgtcgagggatacggacgcaaactcgaacctcccttcccctctctccacagcagcggagctcgtcggcaacttcacacgcaagaacctcactgccgaggatatggtcgtcctctccggtgcacatactgtcggacggtcccactgttcgtccttcaccaaccgcttgtatggattctcgaacgcatcggacgtggaccccaccatttcgtcggcctacgcactcttgctccgagccatttgtccttccaacacctcccagttcttccccaacacaactacggatatggacttgattacccctgcgctcttggataaccgatactacgtgggactcgccaacaacctcggtctcttcacatccgatcaggcgttgctcaccaacgcaaccctcaagaagtccgtcgatgccttcgtcaagtccgagtcggcatggaaaaccaagttcgccaagtcgatggtcaaaatgggcaacatcgatgtgttgaccggaacgaaaggtgagatcaggctcaactgtcgggtcatcaactccggctcctcgtcctcgggcttgttccagctccacacagccacagcatcggacgaagaattcgcccacgtggcaaccaactga

SEQ ID NO: 59 - protein, 358 aa, Artificial Sequence
  Synthetic Construct
MKFFTTILSTASLVAALPAAVDSLGAGLKVGFYSKTCPSAESLVQQAVAAAFKNNSGIAAGLIRLHFHDCFVRGCDGSVLIDSTANNTAEKDAVPNNPSLRGFEVIDAAKKAVEARCPKTVSCADILAFAARDSIALAGNNLTYKVPAGRRDGRVSRDTDANSNLPSPLSTAAELVGNFTRKNLTAEDMVVLSGAHTVGRSHCSSFTNRLYGFSNASDVDPTISSAYALLLRAICPSNTSQFFPNTTTDMDLITPALLDNRYYVGLANNLGLFTSDQALLTNATLKKSVDAFVKSESAWKTKFAKSMVKMGNIDVLTGTKGEIRLNCRVINSGSSSSGLFQLHTATASDEEFAHVATN

SEQ ID NO: 60 - DNA, 1107 bp, Artificial Sequence
  Artificial construct
atgagattatcgacttcgagtctcttcctttccgtgtctctgctggggaagctggccctcgggagccctttgccccaacagcagcgatatggcaaacgcctaggtgccggtctcaaagtgggattctactcgaaaacgtgtccctcggcagagtcgctcgtccagcaggccgtcgcagcggcattcaagaacaactcgggcatcgcagccggtttgatccggttgcacttccacgactgtttcgtgcgaggatgtgacggctccgtcttgattgactcgactgccaacaacacagccgaaaaggatgcagtgcccaacaacccgtccttgcgtggtttcgaggtgatcgacgcagccaagaaagcggtggaagcacgctgtcccaagacagtctcctgtgccgacatcttggcattcgcagcacgagactccatcgcactcgcaggcaacaacttgacctacaaagtgcctgcgggacgacgggatggtcgcgtgtcgagggatacggacgcaaactcgaacctcccttcccctctctccacagcagcggagctcgtcggcaacttcacacgcaagaacctcactgccgaggatatggtcgtcctctccggtgcacatactgtcggacggtcccactgttcgtccttcaccaaccgcttgtatggattctcgaacgcatcggacgtggaccccaccatttcgtcggcctacgcactcttgctccgagccatttgtccttccaacacctcccagttcttccccaacacaactacggatatggacttgattacccctgcgctcttggataaccgatactacgtgggactcgccaacaacctcggtctcttcacatccgatcaggcgttgctcaccaacgcaaccctcaagaagtccgtcgatgccttcgtcaagtccgagtcggcatggaaaaccaagttcgccaagtcgatggtcaaaatgggcaacatcgatgtgttgaccggaacgaaaggtgagatcaggctcaactgtcgggtcatcaactccggctcctcgtcctcgggcttgttccagctccacacagccacagcatcggacgaagaattcgcccacgtggcaaccaactga

SEQ ID NO: 61 - protein, 368 aa, Artificial Sequence
  Synthetic Construct
MRLSTSSLFLSVSLLGKLALGSPLPQQQRYGKRLGAGLKVGFYSKTCPSAESLVQQAVAAAFKNNSGIAAGLIRLHFHDCFVRGCDGSVLIDSTANNTAEKDAVPNNPSLRGFEVIDAAKKAVEARCPKTVSCADILAFAARDSIALAGNNLTYKVPAGRRDGRVSRDTDANSNLPSPLSTAAELVGNFTRKNLTAEDMVVLSGAHTVGRSHCSSFTNRLYGFSNASDVDPTISSAYALLLRAICPSNTSQFFPNTTTDMDLITPALLDNRYYVGLANNLGLFTSDQALLTNATLKKSVDAFVKSESAWKTKFAKSMVKMGNIDVLTGTKGEIRLNCRVINSGSSSSGLFQLHTATASDEEFAHVATN

SEQ ID NO: 62 - DNA, 1083 bp, Artificial Sequence
  Artificial construct
atgaagctactctctctgaccggtgtggctggtgtgcttgcgacttgcgttgcagccactcctttggtgaagcgcctaggtgccggtctcaaagtgggattctactcgaaaacgtgtccctcggcagagtcgctcgtccagcaggccgtcgcagcggcattcaagaacaactcgggcatcgcagccggtttgatccggttgcacttccacgactgtttcgtgcgaggatgtgacggctccgtcttgattgactcgactgccaacaacacagccgaaaaggatgcagtgcccaacaacccgtccttgcgtggtttcgaggtgatcgacgcagccaagaaagcggtggaagcacgctgtcccaagacagtctcctgtgccgacatcttggcattcgcagcacgagactccatcgcactcgcaggcaacaacttgacctacaaagtgcctgcgggacgacgggatggtcgcgtgtcgagggatacggacgcaaactcgaacctcccttcccctctctccacagcagcggagctcgtcggcaacttcacacgcaagaacctcactgccgaggatatggtcgtcctctccggtgcacatactgtcggacggtcccactgttcgtccttcaccaaccgcttgtatggattctcgaacgcatcggacgtggaccccaccatttcgtcggcctacgcactcttgctccgagccatttgtccttccaacacctcccagttcttccccaacacaactacggatatggacttgattacccctgcgctcttggataaccgatactacgtgggactcgccaacaacctcggtctcttcacatccgatcaggcgttgctcaccaacgcaaccctcaagaagtccgtcgatgccttcgtcaagtccgagtcggcatggaaaaccaagttcgccaagtcgatggtcaaaatgggcaacatcgatgtgttgaccggaacgaaaggtgagatcaggctcaactgtcgggtcatcaactccggctcctcgtcctcgggcttgttccagctccacacagccacagcatcggacgaagaattcgcccacgtggcaaccaactga

SEQ ID NO: 63 - protein, 360 aa, Artificial Sequence
  Synthetic Construct
MKLLSLTGVAGVLATCVAATPLVKRLGAGLKVGFYSKTCPSAESLVQQAVAAAFKNNSGIAAGLIRLHFHDCFVRGCDGSVLIDSTANNTAEKDAVPNNPSLRGFEVIDAAKKAVEARCPKTVSCADILAFAARDSIALAGNNLTYKVPAGRRDGRVSRDTDANSNLPSPLSTAAELVGNFTRKNLTAEDMVVLSGAHTVGRSHCSSFTNRLYGFSNASDVDPTISSAYALLLRAICPSNTSQFFPNTTTDMDLITPALLDNRYYVGLANNLGLFTSDQALLTNATLKKSVDAFVKSESAWKTKFAKSMVKMGNIDVLTGTKGEIRLNCRVINSGSSSSGLFQLHTATASDEEFAHVATN

SEQ ID NO: 64 - DNA, 1065 bp, Artificial Sequence
  Artificial construct
atgaagctactctctctgaccggtgtggctggtgtgcttgcgacttgcgttgcagccctaggtgccggtctcaaagtgggattctactcgaaaacgtgtccctcggcagagtcgctcgtccagcaggccgtcgcagcggcattcaagaacaactcgggcatcgcagccggtttgatccggttgcacttccacgactgtttcgtgcgaggatgtgacggctccgtcttgattgactcgactgccaacaacacagccgaaaaggatgcagtgcccaacaacccgtccttgcgtggtttcgaggtgatcgacgcagccaagaaagcggtggaagcacgctgtcccaagacagtctcctgtgccgacatcttggcattcgcagcacgagactccatcgcactcgcaggcaacaacttgacctacaaagtgcctgcgggacgacgggatggtcgcgtgtcgagggatacggacgcaaactcgaacctcccttcccctctctccacagcagcggagctcgtcggcaacttcacacgcaagaacctcactgccgaggatatggtcgtcctctccggtgcacatactgtcggacggtcccactgttcgtccttcaccaaccgcttgtatggattctcgaacgcatcggacgtggaccccaccatttcgtcggcctacgcactcttgctccgagccatttgtccttccaacacctcccagttcttccccaacacaactacggatatggacttgattacccctgcgctcttggataaccgatactacgtgggactcgccaacaacctcggtctcttcacatccgatcaggcgttgctcaccaacgcaaccctcaagaagtccgtcgatgccttcgtcaagtccgagtcggcatggaaaaccaagttcgccaagtcgatggtcaaaatgggcaacatcgatgtgttgaccggaacgaaaggtgagatcaggctcaactgtcgggtcatcaactccggctcctcgtcctcgggcttgttccagctccacacagccacagcatcggacgaagaattcgcccacgtggcaaccaactga

SEQ ID NO: 65 - protein, 354 aa, Artificial Sequence
  Synthetic Construct
MKLLSLTGVAGVLATCVAALGAGLKVGFYSKTCPSAESLVQQAVAAAFKNNSGIAAGLIRLHFHDCFVRGCDGSVLIDSTANNTAEKDAVPNNPSLRGFEVIDAAKKAVEARCPKTVSCADILAFAARDSIALAGNNLTYKVPAGRRDGRVSRDTDANSNLPSPLSTAAELVGNFTRKNLTAEDMVVLSGAHTVGRSHCSSFTNRLYGFSNASDVDPTISSAYALLLRAICPSNTSQFFPNTTTDMDLITPALLDNRYYVGLANNLGLFTSDQALLTNATLKKSVDAFVKSESAWKTKFAKSMVKMGNIDVLTGTKGEIRLNCRVINSGSSSSGLFQLHTATASDEEFAHVATN

SEQ ID NO: 66 - DNA, 972 bp, Nicotiana tabacum
  CDS (1)..(972)
atgtcgttcctccgcttcgtgggagcgatcctcttcctcgtcgcgatcttcggagcgtcgaacgcccagttgtccgcgactttctatgataccacttgtcccaacgtgacatcgatcgtgcgtggcgtcatggaccagaggcagcgcacggatgcgcgagccggtgccaaaatcatccgattgcatttccatgactgtttcgtgaacggctgtgacggctcgatcttgctcgacacagacggtacgcagaccgagaaggatgcccctgccaacgtcggagcgggtggtttcgacatcgtggacgatatcaaaactgccttggagaacgtctgtcctggcgtcgtctcctgtgccgacatcctcgcgctcgcctcggaaatcggcgtggtgctcgcgaaaggaccctcgtggcaggtcttgttcggcaggaaggactcgttgactgccaacaggtccggagccaactcggacatcccctcgcccttcgagacgttggccgtcatgatccctcagttcaccaacaagggcatggacctcaccgacttggtcgcgttgtcgggagcccacaccttcggaagggccaggtgtggcaccttcgagcagcgactcttcaacttcaacggctcgggtaaccccgatttgaccgtggacgccactttcctccagacattgcagggcatctgtccccagggtggaaacaacggcaacacgttcacgaacctcgacatctccactccgaacgacttcgacaacgactacttcaccaacttgcagtcgaaccagggcctcttgcagacggatcaggagttgttctcgacatccggttccgccacaattgcaattgtcaacaggtatgcaggctcgcagacacagttcttcgatgatttcgtgtcgtccatgatcaagctcggtaacatttcgcctctcaccggtaccaacggccagatcaggaccgattgtaagcgcgtgaac

SEQ ID NO: 67 - protein, 324 aa, Nicotiana tabacum
MSFLRFVGAILFLVAIFGASNAQLSATFYDTTCPNVTSIVRGVMDQRQRTDARAGAKIIRLHFHDCFVNGCDGSILLDTDGTQTEKDAPANVGAGGFDIVDDIKTALENVCPGVVSCADILALASEIGVVLAKGPSWQVLFGRKDSLTANRSGANSDIPSPFETLAVMIPQFTNKGMDLTDLVALSGAHTFGRARCGTFEQRLFNFNGSGNPDLTVDATFLQTLQGICPQGGNNGNTFTNLDISTPNDFDNDYFTNLQSNQGLLQTDQELFSTSGSATIAIVNRYAGSQTQFFDDFVSSMIKLGNISPLTGTNGQIRTDCKRVN

SEQ ID NO: 68 - protein, 7 aa, Artificial Sequence
  Motif
GCDXSXL

SEQ ID NO: 69 - protein, 8 aa, Artificial Sequence
  Motif
GCDXSXXX

SEQ ID NO: 70 - protein, 7 aa, Artificial Sequence
  Motif
VSCXDXX

(19) United States
(12) Patent Application Publication
(10) Pub. No.: US 2013/0302878 A1
(43) Pub. Date: Nov. 14, 2013

(54) EXPRESSION OF PLANT PEROXIDASES IN FILAMENTOUS FUNGI

(71) Applicants: Lars Henrik Oestergaard, Charlottenlund (DK); Lisbeth Kalum, Vaerloese (DK)

(72) Inventors: Lars Henrik Oestergaard, Charlottenlund (DK); Lisbeth Kalum, Vaerloese (DK)

(73) Assignee: NOVOZYMES A/S, Bagsvaerd (DK)

(21) Appl. No.: 13/980,347

(22) PCT Filed: Jan. 20, 2012
(86) PCT No.: PCT/EP2012/050910

§ 371 Date: Jul. 18, 2013

(30) Foreign Application Priority Data
Jan. 20, 2011 (EP) 11151562.3

(51) Int. Cl. C12N 9/08

(52) U.S. Cl. CPC C12N 9/0065
USPC 435/192; 536/23.2; 435/254.3

(57) ABSTRACT

The present invention relates to recombinant expression of plant derived peroxidases in filamentous fungal host organisms.

### REFERENCE TO A SEQUENCE LISTING

This application contains a Sequence Listing in computer readable form, which is incorporated herein by reference.

### BACKGROUND OF THE INVENTION

1. Field of the Invention

The present invention relates to methods and compositions for recombinant expression of wildtype plant peroxidases, or peroxidases derived therefrom, in filamentous fungal host organisms.

2. Description of the Related Art

Peroxidases and laccases are well-known enzymes belonging to the group of oxidoreductases. Peroxidases belong to enzyme class EC 1.11.1.7, and laccases belong to EC 1.10.3.2. Both enzyme classes are capable of oxidizing substrates, and therefore they are often used in bleaching applications. Commercial applications include bleaching of denim (abraded look on jeans), bleaching of rinse water after a textile dyeing process, and dye transfer inhibition during a laundering process.

Usually plant peroxidases are purified from plants, but this is a complex process with low yields. Alternatively, recombinant expression in bacteria or yeast can be used, but this also results in poor yields. The need for efficient recombinant production of peroxidases and laccases is thus apparent.

However, the scientific literature is absent of examples showing expression of oxidoreductases derived from plants, in filamentous fungi like Aspergillus. Aspergillus sp. and other filamentous fungi are often used as highly efficient expression hosts for recombinant expression of enzymes. Since researchers rarely report in the literature what does not work, it is believed that the lack of successful examples of oxidoreductase expression illustrates, that it is not considered possible (a technical prejudice) to express plant-derived oxidoreductases in filamentous fungi.

The assumption that plant-derived oxidoreductases cannot be expressed in e.g. Aspergillus sp., is supported by the fact that the inventors of the present invention earlier unsuccessfully attempted expression of a number of plant derived laccases in Aspergillus sp.

### SUMMARY OF THE INVENTION

The inventors of the present invention have found that it is indeed possible expressing plant peroxidases in Aspergillus host cells. Accordingly, the present invention provides methods for recombinant expression of wildtype plant peroxidases, or peroxidases derived therefrom, comprising expressing in a filamentous fungal host organism a nucleic acid sequence encoding a peroxidase, wherein the amino acid sequence of the peroxidase comprises one or more amino acid motifs selected from the group consisting of:

HFHDCFV;
GCD[A, G]S[V, I][I, L][I, L];
and
VSC[A, S]D[I, L][I, L].

### Definitions

Sequence Identity: The relatedness between two amino acid sequences or between two nucleotide sequences is described by the parameter “sequence identity”.

For purposes of the present invention, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Coding sequence: The term “coding sequence” means a polynucleotide, which directly specifies the amino acid sequence of a polypeptide. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic, or recombinant polynucleotide.

cDNA: The term “cDNA” means a DNA molecule that can be prepared by reverse transcription from a mature, spliced, mRNA molecule obtained from a eukaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present invention.

Control sequences: The term “control sequences” means all components necessary for the expression of a polynucleotide encoding a polypeptide of the present invention. Each control sequence may be native or foreign to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Operably linked: The term “operably linked” means a configuration in which a control sequence is placed at an appropriate position relative to the coding sequence of a polynucleotide such that the control sequence directs the expression of the coding sequence.

Expression: The term “expression” includes any step involved in the production of the polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to additional nucleotides that provide for its expression.

Host cell: The term “host cell” or “host organism” means any cell type that is susceptible to transformation, transfection, transduction, and the like with a nucleic acid construct or expression vector comprising a polynucleotide of the present invention. The term “host cell” encompasses any progeny of a parent cell that is not identical to the parent cell due to mutations that occur during replication.

### DETAILED DESCRIPTION OF THE INVENTION

### Peroxidases

EC-numbers may be used for classification of enzymes. Reference is made to the Recommendations of the Nomenclature Committee of the International Union of Biochemistry and Molecular Biology, Academic Press Inc., 1992.

It is to be understood that the term enzyme, as well as the various enzymes and enzyme classes mentioned herein, encompass wild-type enzymes, as well as any variant thereof that retains the activity in question. Such variants may be produced by recombinant techniques. The wild-type enzymes may also be produced by recombinant techniques, or by isolation and purification from the natural source.

In a particular embodiment the enzyme in question is well-defined, meaning that only one major enzyme component is present. This can be inferred e.g. by fractionation on an appropriate size-exclusion column. Such well-defined, or purified, or highly purified, enzyme can be obtained as is known in the art and/or described in publications relating to the specific enzyme in question.

A peroxidase according to the invention is a plant peroxidase enzyme comprised by the enzyme classification EC 1.11.1.7, or any fragment derived therefrom, exhibiting peroxidase activity. Plant peroxidases belong to class III peroxidases.

Class III peroxidases or the secreted plant peroxidases (EC 1.11.1.7) are found only in plants, where they form large multigenic families. Although their primary sequence differs in some points from the classes I and II, their three-dimensional structures are very similar to those of class II, and they also possess calcium ions, disulfide bonds, and an N-terminal signal for secretion.

Class III peroxidases are additionally able to undertake a second cyclic reaction, called hydroxylic, which is distinct from the peroxidative one. During the hydroxylic cycle, peroxidases pass through a Fe(II) state and use mainly the superoxide anion (02) to generate hydroxyl radicals (OH). Class III peroxidases, by using both these cycles, are known to participate in many different plant processes from germination to senescence, for example, auxin metabolism, cell wall elongation and stiffening, or protection against pathogens (see also Passardi et al. “The class III peroxidase multigenic family in rice and its evolution in land plants”, Phytochemistry, 65(13), pp. 1879-93 (2004)).

The amino acid sequence of the peroxidase includes characteristic motifs of plant peroxidases. Preferably, the peroxidase comprises one, two or three amino acid motifs selected from the group consisting of:

(SEQ ID NO: 5)
HFHDCFV;
(SEQ ID NO: 69)
GCD[A, G]S[V, I][I, L][I, L];
and
(SEQ ID NO: 70)
VSC[A, S]D[I, L][I, L].

More preferably, the peroxidase comprises one, two or three amino acid motifs selected from the group consisting of:

(SEQ ID NO: 5)
HFHDCFV;
(SEQ ID NO: 6)
GCD[A, G]S[V, I]LL;
and
(SEQ ID NO: 7)
VSC[A, S]D[I, L]L.

Most preferably, the peroxidase comprises one, two or three amino acid motifs selected from the group consisting of:

(SEQ ID NO: 5)
HFHDCFV;
(SEQ ID NO: 68)
GCD[A, G]S[V, I]L;
and
(SEQ ID NO: 7)
VSC[A, S]D[I, L]L

The peroxidase of the invention comprises an amino acid sequence which has at least 60% identity, such as at least 65% identity, at least 70% identity, at least 75% identity, at least 80% identity, at least 85% identity, at least 90% identity, or at least 95% identity to the amino acid sequence of SEQ ID NO:2, SEQ ID NO:4, amino acids 38 to 354 of SEQ ID NO: 45, amino acids 30 to 362 of SEQ ID NO: 55, or amino acids 23 to 324 of SEQ ID NO: 67.

In an embodiment, the peroxidase consists of an amino acid sequence which has at least 60% identity, such as at least 65% identity, at least 70% identity, at least 75% identity, at least 80% identity, at least 85% identity, at least 90% identity, or at least 95% identity to the amino acid sequence of SEQ ID NO:2, SEQ ID NO:4, amino acids 38 to 354 of SEQ ID NO: 45, amino acids 30 to 362 of SEQ ID NO: 55, or amino acids 23 to 324 of SEQ ID NO: 67.

In another embodiment, the peroxidase may be identical to, or have one or several amino acid differences as compared to, the amino acid sequence of SEQ ID NO:2, SEQ ID NO:4, amino acids 38 to 354 of SEQ ID NO: 45, amino acids 30 to 362 of SEQ ID NO: 55, or amino acids 23 to 324 of SEQ ID NO: 67; such as at the most 10 amino acid differences; or at the most 9, 8, 7, 6, 5, 4, 3, 2, or 1 amino acid difference(s), as compared to the amino acid sequence of SEQ ID NO:2, SEQ ID NO:4, amino acids 38 to 354 of SEQ ID NO: 45, amino acids 30 to 362 of SEQ ID NO: 55, or amino acids 23 to 324 of SEQ ID NO: 67.

Preferably, the peroxidase of the invention is a soybean peroxidase (e.g. SEQ ID NO:2) or is derived from a soybean peroxidase; or a royal palm tree peroxidase (e.g. SEQ ID NO:4) or is derived from a royal palm tree peroxidase; or a poplar peroxidase (e.g. amino acids 38 to 354 of SEQ ID NO: 45) or is derived from a poplar peroxidase; or a maize peroxidase (e.g. amino acids 30 to 362 of SEQ ID NO: 55) or is derived from a maize peroxidase; or a tobacco peroxidase (e.g. amino acids 23 to 324 of SEQ ID NO: 67) or is derived from a tobacco peroxidase.

### Determination of Peroxidase Activity (PDXU)

One peroxidase unit (PDXU) is the amount of enzyme which catalyze the conversion of one μmole hydrogen peroxide per minute at 30° C. in an aqueous solution of:
0.1 M phosphate buffer, pH 7.0;
0.88 mM hydrogen peroxide; and
1.67 mM 2,2′-azino-bis(3-ethylbenzothiazoline-6-sulfonate) (ABTS).

The reaction is continued for 60 seconds (15 seconds after mixing) while the change in absorbance at 418 nm is measured. The absorbance should be in the range of 0.15 to 0.30. Peroxidase activity is calculated using an absorption coefficient of oxidized ABTS of 36 mM⁻¹ cm⁻¹, and a stoichiometry of one μmole H₂O₂ converted per two μmole ABTS oxidized.

### Methods and Uses of the Invention

Commonly, plant peroxidases are purified from plants, but this is a complex process with low yields. Alternatively, recombinant expression in bacteria or yeast can be used, but this often results in poor yields and/or difficult purification. The need for efficient recombinant production of plant derived peroxidases is thus apparent.

According to the present invention, wildtype plant peroxidases, and peroxidases derived therefrom, can be produced as recombinant protein in a filamentous fungal host cell, which often solves the problem of poor yields and/or difficult purification.

Recombinant expression of proteins is not always straight forward and it is hard to predict whether the desired product can in fact be produced in a particular production host organism and whether product yields will be sufficient for establishing an economical production.

Several parameters can lead to a lack of expression in filamentous fungal hosts. In general expression of a secreted and correctly processed peroxidase in a filamentous fungus involves a number of steps any of which could be a limiting step.

First the inserted peroxidase gene is transcribed to hnRNA. Then the hnRNA is transported from the nucleus to the cytosol, and during this process it is maturated to mRNA. Generally, a mRNA pool is established in the cytosol in order to sustain translation. The mRNA is then translated to a protein precursor, and this precursor is subsequently secreted to the endoplasmatic reticulum (ER) either co-translationally or post-translationally. Upon translocation into the ER the secretion signal peptide is cleaved of by a signal peptidase, and the resulting protein is folded in the ER. Secretion of the protein to the golgi apparatus follows when proper folding has been recognized by the cell. Here the propeptide will be cleaved to release the mature peroxidase. Thus numerous possibilities exist for preventing sufficient expression of a gene sequence in a given host organism.

In order to provide efficient expression of a polynucleotide sequence encoding a desired protein the translation process has to be efficient. One object of the present invention is therefore to optimize the mRNA sequence encoding the peroxidase protein in order to obtain sufficient expression in a filamentous fungal host cell.

In one embodiment, the present invention relates to a method for recombinant expression of a wild type plant peroxidase in a filamentous fungal host organism comprising expressing a modified nucleic acid sequence encoding a wild type plant peroxidase in a filamentous fungal host organism, wherein the modified nucleic acid sequence differs in at least one codon from the wild type nucleic acid sequence encoding the wild type plant peroxidase.

The modified nucleic acid sequence may be obtained by a) providing a wild type nucleic acid sequence encoding a wild type plant peroxidase and b) modifying at least one codon of said nucleic acid sequence so that the modified nucleic acid sequence differs in at least one codon from each wild type nucleic acid sequence encoding the wild type plant peroxidase. Methods for modifying nucleic acid sequences are well known to a person skilled in the art. In a particular embodiment said modification does not change the identity of the amino acid encoded by said codon.

Thus in another aspect the object of the present invention is provided by a method for recombinant expression of a wild type plant peroxidase in a filamentous fungal host organism, comprising the steps:
i) providing a nucleic acid sequence encoding a wild type plant peroxidase, said nucleic acid sequence comprising at least one modified codon, wherein the modification does not change the amino acid encoded by said codon and the nucleic acid sequence of said codon is different compared to the corresponding codon in the nucleic acid sequence encoding the wild type gene;
ii) expressing the modified nucleic acid sequence in the filamentous fungal host.

The starting nucleic acid sequence to be modified according to this embodiment is a wild type nucleic acid sequence encoding the plant peroxidase of interest.

Modifications according to the invention, comprises any modification of the base triplet and in a particular embodiment they comprise any modification which does not change the identity of the amino acid encoded by said codon, i.e. the amino acid encoded by the original codon and the modified codon is the same. In most cases the modification will be at the third position, however, in a few cases the modification may also be at the first or the second position. How to modify a codon also without modifying the resulting amino acid is known to the skilled person.

For both of the above embodiments, the number of codon which should differ or the number of modifications needed in order to obtain sufficient expression may vary. Thus according to a further embodiment of the invention, the modified nucleic acid sequence differs in at least 2 codons from each wild type nucleic acid sequence encoding said wild type plant peroxidase or at least 2 codons have been modified, particularly at least 3 codons, more particularly at least 5 codons, more particularly at least 10 codons, more particularly at least 15 codons, even more particularly at least 25 codons.

It has furthermore been found, that by changing the codon usage of the wild type nucleic acid sequence to be selected among the codons preferably used by the filamentous fungus used as a host, the expression of a peroxidase of the invention is now possible. Such codons are said to be “optimized” for expression.

Due to the degeneracy of the genetic code and the preference of certain preferred codons in particular organisms/cells the expression level of a protein in a given host cell can in some instances be improved by optimizing the codon usage. In the present case, the yields of plant peroxidase were excellent when the wild type nucleic acid sequences encoding SEQ ID NO: 2, SEQ ID NO: 4, amino acids 38 to 354 of SEQ ID NO: 45, amino acids 30 to 362 of SEQ ID NO: 55, and amino acids 23 to 324 of SEQ ID NO: 67 were optimized by codon optimization and expressed in Aspergillus.

In the present invention “codon optimized” means that due to the degeneracy of the genetic code more than one triplet codon can be used for each amino acid. Some codons will be preferred in a particular organism and by changing the codon usage in a wild type gene to a codon usage preferred in a particular expression host organism the codons are said to be optimized. Codon optimization can be performed e.g. as described in Gustafsson et al., 2004, (Trends in Biotechnology vol. 22 (7); Codon bias and heterologous protein expression), and U.S. Pat. No. 6,818,752.

Codon optimization may be based on the average codon usage for the host organism or it can be based on the codon usage for a particular gene which is known to be expressed in high amounts in a particular host cell.

In one embodiment of the invention the peroxidase protein is encoded by a modified nucleic acid sequence codon optimized in at least 10% of the codons, more particularly at least 20%, or at least 30%, or at least 40%, or particularly at least 50%, more particularly at least 60%, and more particularly at least 75%. Thus the modified nucleic acid sequence may differ in at least 10% of the codons from each wild type nucleic acid sequence encoding said wild type peroxidase, more particularly in at least 20%, or in at least 30%, or in at least 40%, or particularly in at least 50%, more particularly in at least 60%, and more particularly in at least 75%. In particular said codons may differ because they have been codon optimized as compared with a wild type nucleic acid sequence encoding a wild type plant peroxidase.

Particularly 100% of the nucleic acid sequence has been codon optimized to match the preferred codons used in filamentous fungi.

In a particular embodiment the codon optimization is based on the codon usage of alpha amylase from Aspergillus oryzae, also known as Fungamyl™ (WO 2005/019443; SEQ ID NO: 2), which is a protein known to be expressed in high levels in filamentous fungi. In the present context an expression level corresponding to at least 20%, preferably at least 30%, more preferably at least 40%, even more preferably at least 50%, of the total amount of secreted protein constitutes the protein of interest is considered a high level of expression.

In a particular embodiment, the modified nucleic acid sequence encoding a mature plant peroxidase is selected from the group consisting of SEQ ID NO: 1, SEQ ID NO: 3, amino acids 118 to 1068 of SEQ ID NO: 44, amino acids 94 to 1092 of SEQ ID NO: 54, or amino acids 67 to 972 of SEQ ID NO: 66.

In practice the optimization according to the invention comprises the steps:
i) the nucleic acid sequence encoding the peroxidase of the invention is codon optimized as explained in more detail below;
ii) check the resulting modified sequence for a balanced GC-content (approximately 45-55%); and
iii) check or edit the resulting modified sequence from step ii) as explained below.

### Codon Optimization Protocol:

The codon usage of a single gene, a number of genes or a whole genome can be calculated with the program cusp from the EMBOSS-package (http://www.rfcgr.mrc.ac.uk/Software/EMBOSS/).

The starting point for the optimization is the amino acid sequence of the protein or a nucleic acid sequence coding for the protein together with a codon-table. By a codon-optimized gene, we understand a nucleic acid sequence, encoding a given protein sequence and with the codon statistics given by a codon table.

The codon statistics referred to is a column in the codon-table called “Fract” in the output from cusp-program and which describes the fraction of a given codon among the other synonymous codons. We call this the local score. If for instance 80% of the codons coding for F is TTC and 20% of the codons coding for F are TTT, then the codon TTC has a local score of 0.8 and TTT has a local score of 0.2.

The codons in the codon table are re-ordered by first encoding amino acid (e.g. alphabetically) and then increasingly by the score. In the example above, ordering the codons for F as TTT, TTC. Cumulated scores for the codons are then generated by adding the scores in order. In the example above TTT has a cumulated score of 0.2 and TTC has a cumulated score of 1. The most used codon will always have a cumulated score of 1.

In order to generate a codon optimized gene the following is performed. For each position in the amino acid sequence, a random number between 0 and 1 is generated. This is done by the random-number generator on the computer system on which the program runs. The first codon is chosen as the codon with a cumulated score greater than or equal to the generated random number. If, in the example above, a particular position in the gene is “F” and the random number generator gives 0.5, TTC is chosen as codon.

The strategy for avoiding introns is to make sure that there are no branch points. This was done by making sure that the consensus sequence for branch-point in Aspergillus oryzae: CT[AG]A[CT] was not present in the sequence. The sequence [AG]CT[AG]A[AG] may be recognised as a branch point in introns. Thus in a particular embodiment of the present invention such sequences may also be modified or be removed according to a method of the present invention. This was done in a post processing step, where the sequence was scanned for the presence of this motif, and each occurrence was removed by changing codons in the motif to synonymous codons, choosing codons with the best local score first.

A codon table showing the codon usage of the alpha amylase from Aspergillus oryzae is given below.

TABLE 1
Codon usage for the Aspergillus oryzae alpha amylase
(CUSP codon usage file)

| Codon | Amino acid | Fract | /1000 | Number |
|---|---|---|---|---|
| GCA | A | 0.286 | 24.000 | 12 |
| GCC | A | 0.357 | 30.000 | 15 |
| GCG | A | 0.238 | 20.000 | 10 |
| GCT | A | 0.119 | 10.000 | 5 |
| TGC | C | 0.222 | 4.000 | 2 |
| TGT | C | 0.778 | 14.000 | 7 |
| GAC | D | 0.524 | 44.000 | 22 |
| GAT | D | 0.476 | 40.000 | 20 |
| GAA | E | 0.417 | 10.000 | 5 |
| GAG | E | 0.583 | 14.000 | 7 |
| TTC | F | 0.800 | 24.000 | 12 |
| TTT | F | 0.200 | 6.000 | 3 |
| GGA | G | 0.233 | 20.000 | 10 |
| GGC | G | 0.419 | 36.000 | 18 |
| GGG | G | 0.116 | 10.000 | 5 |
| GGT | G | 0.233 | 20.000 | 10 |
| CAC | H | 0.571 | 8.000 | 4 |
| CAT | H | 0.429 | 6.000 | 3 |
| ATA | I | 0.071 | 4.000 | 2 |
| ATC | I | 0.679 | 38.000 | 19 |
| ATT | I | 0.250 | 14.000 | 7 |
| AAA | K | 0.350 | 14.000 | 7 |
| AAG | K | 0.650 | 26.000 | 13 |
| CTA | L | 0.081 | 6.000 | 3 |
| CTC | L | 0.351 | 26.000 | 13 |
| CTG | L | 0.162 | 12.000 | 6 |
| CTT | L | 0.108 | 8.000 | 4 |
| TTA | L | 0.027 | 2.000 | 1 |
| TTG | L | 0.270 | 20.000 | 10 |
| ATG | M | 1.000 | 22.000 | 11 |
| AAC | N | 0.885 | 46.000 | 23 |
| AAT | N | 0.115 | 6.000 | 3 |
| CCA | P | 0.136 | 6.000 | 3 |
| CCC | P | 0.364 | 16.000 | 8 |
| CCG | P | 0.227 | 10.000 | 5 |
| CCT | P | 0.273 | 12.000 | 6 |
| CAA | Q | 0.250 | 10.000 | 5 |
| CAG | Q | 0.750 | 30.000 | 15 |
| AGA | R | 0.000 | 0.000 | 0 |
| AGG | R | 0.300 | 6.000 | 3 |
| CGA | R | 0.200 | 4.000 | 2 |
| CGC | R | 0.200 | 4.000 | 2 |
| CGG | R | 0.200 | 4.000 | 2 |
| CGT | R | 0.100 | 2.000 | 1 |
| AGC | S | 0.162 | 12.000 | 6 |
| AGT | S | 0.108 | 8.000 | 4 |
| TCA | S | 0.108 | 8.000 | 4 |
| TCC | S | 0.243 | 18.000 | 9 |
| TCG | S | 0.270 | 20.000 | 10 |
| TCT | S | 0.108 | 8.000 | 4 |
| ACA | T | 0.250 | 20.000 | 10 |
| ACC | T | 0.325 | 26.000 | 13 |
| ACG | T | 0.200 | 16.000 | 8 |
| ACT | T | 0.225 | 18.000 | 9 |
| GTA | V | 0.129 | 8.000 | 4 |
| GTC | V | 0.387 | 24.000 | 12 |
| GTG | V | 0.323 | 20.000 | 10 |
| GTT | V | 0.161 | 10.000 | 5 |
| TGG | W | 1.000 | 24.000 | 12 |
| TAC | Y | 0.686 | 48.000 | 24 |
| TAT | Y | 0.314 | 22.000 | 11 |
| TAA | * | 0.000 | 0.000 | 0 |
| TAG | * | 0.000 | 0.000 | 0 |
| TGA | * | 1.000 | 2.000 | 1 |

### Introns

Eukaryotic genes may be interrupted by intervening sequences (introns) which must be modified in precursor transcripts in order to produce functional mRNAs. This process of intron removal is known as pre-mRNA splicing. Usually, a branchpoint sequence of an intron is necessary for intron splicing through the formation of a lariat. Signals for splicing reside directly at the boundaries of the intron splice sites. The boundaries of intron splice sites usually have the consensus intron sequences GT and AG at their 5′ and 3′ extremities, respectively. While no 3′ splice sites other than AG have been reported, there are reports of a few exceptions to the 5′ GT splice site. For example, there are precedents where CT or GC is substituted for GT at the 5′ boundary. There is also a strong preference for the nucleotide bases ANGT to follow GT where N is A, C, G, or T (primarily A or T in Saccharomyces species), but there is no marked preference for any particular nucleotides to precede the GT splice site. The 3′ splice site AG is primarily preceded by a pyrimidine nucleotide base (Py), i.e., C or T.

The number of introns that can interrupt a fungal gene ranges from one to twelve or more introns (Rymond and Rosbash, 1992, In, E. W. Jones, J. R. Pringle, and J. R. Broach, editors, The Molecular and Cellular Biology of the Yeast Saccharomyces, pages 143-192, Cold Spring Harbor Laboratory Press, Plainview, N.Y.; Gurr et al., 1987, In Kinghorn, J. R. (ed.), Gene Structure in Eukaryotic Microbes, pages 93-139, IRL Press, Oxford). They may be distributed throughout a gene or situated towards the 5′ or 3′ end of a gene. In Saccharomyces cerevisiae, introns are located primarily at the 5′ end of the gene. Introns may be generally less than 1 kb in size, and usually are less than 400 by in size in yeast and less than 100 by in filamentous fungi.

The Saccharomyces cerevisiae intron branchpoint sequence 5′-TACTAAC-3′ rarely appears in filamentous fungal introns (Gurr et al., 1987, supra). Sequence stretches closely or loosely resembling TACTAAC are seen at equivalent points in filamentous fungal introns with a general consensus NRCTRAC where N is A, C, G, or T, and R is A or G. For example, the fourth position T is invariant in both the Neurospora crassa and Aspergillus nidulans putative consensus sequences. Furthermore, nucleotides G, A, and C predominate in over 80% of the positions 3, 6, and 7, respectively, although position 7 in Aspergillus nidulans is more flexible with only 65% C. However, positions 1, 2, 5, and 8 are much less strict in both Neurospora crassa and Aspergillus nidulans. Other filamentous fungi have similar branchpoint stretches at equivalent positions in their introns, but the sampling is too small to discern any definite trends.

The heterologous expression of a gene encoding a polypeptide in a fungal host strain may result in the host strain incorrectly recognizing a region within the coding sequence of the gene as an intervening sequence or intron. For example, it has been found that intron-containing genes of filamentous fungi are incorrectly spliced in Saccharomyces cerevisiae (Gurr et al., 1987, In Kinghorn, J. R. (ed.), Gene Structure in Eukaryotic Microbes, pages 93-139, IRL Press, Oxford). Since the region is not recognized as an intron by the parent strain from which the gene was obtained, the intron is called a cryptic intron. This improper recognition of an intron, referred to herein as a cryptic intron, may lead to aberrant splicing of the precursor mRNA molecules resulting in no production of biologically active polypeptide or in the production of several populations of polypeptide products with varying biological activity.

“Cryptic intron” is defined herein as a region of a coding sequence that is incorrectly recognized as an intron which is excised from the primary mRNA transcript. A cryptic intron preferably has 10 to 1500 nucleotides, more preferably 20 to 1000 nucleotides, even more preferably 30 to 300 nucleotides, and most preferably 30 to 100 nucleotides.

The presence of cryptic introns can in particular be a problem when trying to express proteins in organisms which have a less strict requirement to what sequences are necessary in order to define an intron. Such “sloppy” recognition can result e.g. when trying to express recombinant proteins in fungal expression systems.

Cryptic introns can be identified by the use of Reverse Transcription Polymerase Chain Reaction (RT-PCR). In RT_PCR, mRNA is reverse transcribed into single stranded cDNA that can be PCR amplified to double stranded cDNA. PCR primers can then be designed to amplify parts of the single stranded or double stranded cDNA, and sequence analysis of the resulting PCR products compared to the sequence of the genomic DNA reveals the presence and exact location of cryptic introns (T. Kumazaki et al. (1999) J. Cell. Sci. 112, 1449-1453).

According to one embodiment of the invention the modification introduced into the wild type gene sequence will optimize the mRNA for expression in a particular host organism. In the present invention the host organism or host cell comprises a group of fungi referred to as filamentous fungi as explained in more detail below.

### Filamentous Fungal Host Organism

The host organism (host cell) of the invention is a filamentous fungus represented by the following groups of Ascomycota, include, e.g., Neurospora, Eupenicillium (=Penicillium), Emericella (=Aspergillus), Eurotium (=Aspergillus).

In a preferred embodiment, the filamentous fungus includes all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK). The filamentous fungi are characterized by a vegetative mycelium composed of chitin, cellulose, glucan, chitosan, mannan, and other complex polysaccharides. Vegetative growth is by hyphal elongation and carbon catabolism is obligately aerobic.

In a more preferred embodiment, the filamentous fungal host cell is a cell of a species of, but not limited to, Acremonium, Aspergillus, Fusarium, Humicola, Mucor, Myceliophthora, Neurospora, Penicillium, Thielavia, Tolypocladium, and Trichoderma or a teleomorph or synonym thereof. In an even more preferred embodiment, the filamentous fungal host cell is an Aspergillus cell. In another even more preferred embodiment, the filamentous fungal host cell is an Acremonium cell. In another even more preferred embodiment, the filamentous fungal host cell is a Fusarium cell. In another even more preferred embodiment, the filamentous fungal host cell is a Humicola cell. In another even more preferred embodiment, the filamentous fungal host cell is a Mucor cell. In another even more preferred embodiment, the filamentous fungal host cell is a Myceliophthora cell. In another even more preferred embodiment, the filamentous fungal host cell is a Neurospora cell. In another even more preferred embodiment, the filamentous fungal host cell is a Penicillium cell. In another even more preferred embodiment, the filamentous fungal host cell is a Thielavia cell. In another even more preferred embodiment, the filamentous fungal host cell is a Tolypocladium cell. In another even more preferred embodiment, the filamentous fungal host cell is a Trichoderma cell. In a most preferred embodiment, the filamentous fungal host cell is an Aspergillus awamori, Aspergillus foetidus, Aspergillus japonicus, Aspergillus aculeatus, Aspergillus niger, Aspergillus nidulans or Aspergillus oryzae cell. In another preferred embodiment, the filamentous fungal host cell is a Fusarium cell of the section Discolor (also known as the section Fusarium). For example, the filamentous fungal parent cell may be a Fusarium bactridioides, Fusarium cerealis, Fusarium crookwellense, Fusarium culmorum, Fusarium graminearum, Fusarium graminum, Fusarium heterosporum, Fusarium negundi, Fusarium reticulatum, Fusarium roseum, Fusarium sambucinum, Fusarium sarcochroum, Fusarium sulphureum, or Fusarium trichothecioides cell. In another preferred embodiment, the filamentous fungal parent cell is a Fusarium strain of the section Elegans, e.g., Fusarium oxysporum. In another most preferred embodiment, the filamentous fungal host cell is a Humicola insolens or Humicola lanuginosa cell. In another most preferred embodiment, the filamentous fungal host cell is a Mucor miehei cell. In another most preferred embodiment, the filamentous fungal host cell is a Myceliophthora thermophilum cell. In another most preferred embodiment, the filamentous fungal host cell is a Neurospora crassa cell. In another most preferred embodiment, the filamentous fungal host cell is a Penicillium purpurogenum or Penicillium funiculosum (WO 00/68401) cell. In another most preferred embodiment, the filamentous fungal host cell is a Thielavia terrestris cell. In another most preferred embodiment, the Trichoderma cell is a Trichoderma harzianum, Trichoderma koningii, Trichoderma longibrachiatum, Trichoderma reesei or Trichoderma viride cell.

In a particular embodiment the filamentous host cell is an A. oryzae or A. niger cell.

In a preferred embodiment of the invention the host cell is a protease deficient or protease minus strain.

This may e.g. be the protease deficient strain Aspergillus oryzae JaL 125 having the alkaline protease gene named “alp” deleted. This strain is described in WO 97/35956 (Novozymes), or EP patent no. 429,490, or the TPAP free host cell, in particular a strain of A. niger, disclosed in WO 96/14404. Further, also host cell, especially A. niger or A. oryzae, with reduced production of the transcriptional activator (prtT) as described in WO 01/68864 is specifically contemplated according to the invention.

### Transformation of Fungi

Fungal cells may be transformed by a process involving protoplast formation, transformation of the protoplasts, and regeneration of the cell wall in a manner known per se. Suitable procedures for transformation of Aspergillus host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156 and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

### Methods of Production

The present invention also relates to expression of the modified nucleic acid sequence in order to produce the peroxidase of the invention. Expression comprises (a) cultivating a filamentous fungus expressing the peroxidase from the modified nucleic acid sequence; and (b) recovering the peroxidase. Preferably, the filamentous fungus is of the genus Aspergillus, and more preferably Aspergillus oryzae or Aspergillus niger.

In the production methods of the present invention, the cells are cultivated in a nutrient medium suitable for production of the polypeptide using methods known in the art. For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptides may be detected using methods known in the art that are specific for the polypeptides, such as N-terminal sequencing of the polypeptide. These detection methods may include use of specific antibodies. The resulting polypeptide may be recovered by methods known in the art. For example, the polypeptide may be recovered from the nutrient medium by conventional procedures including, but not limited to, centrifugation, filtration, extraction, spray-drying, evaporation, or precipitation.

The polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989).

In a further aspect the present invention relates to a modified nucleic acid sequence encoding a wildtype plant peroxidase, such as soy bean peroxidase (e.g. SEQ ID NO:2), royal palm tree peroxidase (e.g. SEQ ID NO:4), poplar peroxidase (e.g. amino acids 38 to 354 of SEQ ID NO: 45), maize peroxidase (e.g. amino acids 30 to 362 of SEQ ID NO: 55), or tobacco peroxidase (e.g. amino acids 23 to 324 of SEQ ID NO: 67), and capable of expression in a filamentous fungal host organism, which modified nucleic acid sequence is obtainable by:
i) providing the wild type nucleic acid sequence encoding the peroxidase;
ii) modifying at least one codon, wherein the modification does not change the amino acid encoded by said codon and the nucleic acid sequence of said codon is different compared to the corresponding codon in the wild type gene.

In the present context the term “capable of expression in a filamentous host” means that the yield of the peroxidase protein should be at least 1.5 mg/l, more particularly at least 2.5 mg/l, more particularly at least 5 mg/l, more particularly at least 10 mg/l, even more particularly at least 20 mg/l, or more particularly 0.5 g/L, or more particularly 1 g/L, or more particularly 5 g/L, or more particularly 10 g/L, or more particularly 20 g/L.

Specific examples of modified nucleic acid sequences encoding a peroxidase of the invention and modified according to the invention in order to provide expression of the peroxidase protein in a filamentous fungal host, like e.g. Aspergillus, are shown in SEQ ID NO: 1 (soy bean peroxidase), SEQ ID NO: 3 (royal palm tree peroxidase), amino acids 118 to 1068 of SEQ ID NO: 44 (poplar peroxidase), amino acids 94 to 1092 of SEQ ID NO: 54 (maize peroxidase), and amino acids 67 to 972 of SEQ ID NO: 66 (tobacco peroxidase). The information disclosed herein will allow the skilled person to isolate other modified nucleic acid sequences following the directions above, which sequences can also be expressed in filamentous fungi and such sequences are also comprised within the scope of the present invention.

### Methods and Compositions

In a first aspect, the present invention provides a method for recombinant expression of a plant peroxidase, comprising expressing in a filamentous fungal host organism a nucleic acid sequence encoding a peroxidase, wherein the amino acid sequence of the peroxidase comprises one, two or three amino acid motifs selected from the group consisting of:

HFHDCFV;
GCD[A, G]S[V, I][I, L][I, L];
and
VSC[A, S]D[I, L][I, L].

Preferably, the motifs are selected from the group consisting of:

HFHDCFV;
GCD[A, G]S[V, I]LL;
and
VSC[A, S]D[I, L]L.

In an embodiment, the peroxidase is a class III peroxidase from EC 1.11.1.7

In another embodiment, the amino acid sequence of the peroxidase has at least 65% identity, preferably at least 70% identity, at least 75% identity, at least 80% identity, at least 85% identity, at least 90% identity, or at least 95% identity, to the amino acid sequence of SEQ ID NO:2, SEQ ID NO:4, amino acids 38 to 354 of SEQ ID NO: 45, amino acids 30 to 362 of SEQ ID NO: 55, or amino acids 23 to 324 of SEQ ID NO: 67.

In another embodiment, the peroxidase consists of the amino acid sequence of SEQ ID NO:2, SEQ ID NO:4, amino acids 38 to 354 of SEQ ID NO: 45, amino acids 30 to 362 of SEQ ID NO: 55, or amino acids 23 to 324 of SEQ ID NO: 67.

The nucleic acid sequence may be attached to suitable control sequence(s) that provide for expression of the peroxidase.

In another embodiment, at least one codon of the nucleic acid sequence is optimized for translation in a filamentous fungal host organism. Preferably, at least half of the codons of the nucleic acid sequence are optimized for translation in a filamentous fungal host organism. More preferably, the nucleic acid sequence is codon optimized in at least 10% of the codons, preferably at least 20% of the codons, more preferably at least 30% of the codons, more preferably at least 50% of the codons, and most preferably at least 75% of the codons. Most preferably, the optimized codon(s) corresponds to the codon usage of alpha amylase from Aspergillus oryzae.

In another embodiment, the filamentous fungal host organism is selected from the group consisting of Acremonium, Aspergillus, Fusarium, Humicola, Mucor, Myceliophthora, Neurospora, Penicillium, Thielavia, Tolypocladium, or Trichoderma. Preferably, the filamentous fungal host organism is an Aspergillus sp., more preferably Aspergillus awamori, Aspergillus foetidus, Aspergillus japonicus, Aspergillus niger, Aspergillus nidulans, or Aspergillus oryzae. Most preferably, the filamentous fungal host organism is Aspergillus oryzae or Aspergillus niger.

In a second aspect, the present invention provides a modified nucleic acid sequence encoding a wild type peroxidase and capable of expression in a filamentous fungal host organism, wherein said modified nucleic acid sequence differs in at least one codon from the wild type nucleic acid sequence encoding the wild type peroxidase, and wherein the peroxidase has at least 60% identity to soy bean peroxidase or royal palm tree peroxidase and comprises one, two or three amino acid motifs selected from the group consisting of:

HFHDCFV;
GCD[A, G]S[V, I]LL;
and
VSC[A, S]D[I, L]L.

In an embodiment, the modification of at least one codon is optimized for translation in an Aspergillus host organism. Preferably, the Aspergillus host organism is Aspergillus awamori, Aspergillus foetidus, Aspergillus japonicus, Aspergillus niger, Aspergillus nidulans, or Aspergillus oryzae. More preferably, the Aspergillus host organism is Aspergillus oryzae or Aspergillus niger.

In another embodiment, the codon usage corresponds to the codon usage of alpha amylase from Aspergillus oryzae.

In another embodiment, the modified nucleic acid sequence is shown as SEQ ID NO: 1, 3, 8, 10, 12, 14, 16, 18, 20, 22, 24, 26, 28, 30, 32, 34, 44, 46, 48, 50, 52, 54, 56, 58, 60, 62, 64, or 66.

In a third aspect, the present invention provides a modified nucleic acid sequence encoding a peroxidase and capable of expression in a filamentous fungal host organism, which has at least 50% identity, preferably at least 60% identity, at least 70% identity, at least 80% identity, or at least 90% identity, to a nucleic acid sequence selected from the group consisting of SEQ ID NOs: 1, 3, 8, 10, 12, 14, 16, 18, 20, 22, 24, 26, 28, 30, 32, 34, 44, 46, 48, 50, 52, 54, 56, 58, 60, 62, 64, and 66.

In another aspect, the present invention also provides a recombinant filamentous fungal host organism, comprising the modified nucleic acid sequence of aspect 2 or aspect 3. In an embodiment, the recombinant filamentous fungal host organism is an Aspergillus sp.; preferably, Aspergillus awamori, Aspergillus foetidus, Aspergillus japonicus, Aspergillus niger, Aspergillus nidulans, or Aspergillus oryzae; and more preferably, Aspergillus oryzae or Aspergillus niger.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

The present invention is further described by the following examples that should not be construed as limiting the scope of the invention.

### EXAMPLES

Plasmid pENI2516 was described in WO 2004/069872, Example 2.

Aspergillus oryzae strain ToC1512 was described in WO 2005/070962, Example 11.

Primer 1:
(SEQ ID NO: 36)
5′-TCCTGACCTAGGACAGCTCACACCCACTTTC-3′
Primer 2:
(SEQ ID NO: 37)
5′-ACAGGTCTTAAGTCATTTGGACTGGGCGACG-3′
Primer 3:
(SEQ ID NO: 38)
5′-TGCCCGCCTAGGAGACCTCCAGATTGGATTCTATAAC-3′
Primer 4:
(SEQ ID NO: 39)
5′-ATCATA CTTAAG TTATCAGGAGTTGACCACGGAACAG-3′
Primer 5:
(SEQ ID NO: 40)
5′-TAATCCTAGGTCAGCTCACACCTACCTTCTAC-3′
Primer 6:
(SEQ ID NO: 41)
5′-GGTACCCTTAAGTCAAATCGAC-3′
Primer 7:
(SEQ ID NO: 42)
5′-TAATCCTAGGTGCCGGTCTCAAAGTGGGATTCTAC-3′
Primer 8:
(SEQ ID NO: 43)
5′-ATTACTTAAGTCAGTTGGTTGCCACGTG-3′

### Example 1

### Cloning and Expression of Soybean Peroxidase

A DNA sequence was designed to encode the amino acid sequence of soybean peroxidase (SEQ ID NO:2) using codon optimization as described above. The gene was specifically designed for expression in Aspergillus oryzae, and a restriction site was added at either end to ease cloning. The DNA was subsequently synthezised by a commercial provider.

The synthetic gene encoding the peroxidase was ligated into the multiple cloning site of plasmid pEN12516 as a BamHI-AfllI fragment to generate construct SEQ ID NO:8 using standard technologies of molecular biology. This construct was used as template in a PCR reaction with Primer 1 and Primer 2 resulting in a fragment with approximate size 1095 bp. The PCR product contains restriction sites at either end which allows ligation of an AvrI-AfllI fragment into existing plasmids to generate constructs SEQ ID NOs: 10, 12, 14, 16, 18 and 20. These constructs contain different secretion signal and prepro sequences known to work well in Aspergillus oryzae. All constructed plasmids were initially transformed into E. coli strain Top10 and the inserts were sequenced to confirm nucleotide sequences. The plasmid was subsequently transformed into Aspergillus oryzae strain ToC1512 for expression trials.

The transformed strain of A. oryzae was grown for expression of peroxidase enzyme. Typically, 200 μL of YP growth medium was inoculated with spores from strains grown on sucrose agar added 10 mM NaNO₃. The cultures were grown in a 96 well sterile microplate for 3-4 days at 34° C. without shaking. Expression of peroxidase was confirmed by presence of a band with the correct molecular weight on SDS-PAGE and by ability to bleach indigo carmine in presence of 10-phenothiazinepropionic acid (PPT):
100 μL 100 mM Britton-Robinson buffer pH 6
2 μL 10 mM indigo carmine
4 μL 10 mM PPT (in 96% ethanol)
2 μL 0.3% hydrogen peroxidase
10 μL supernatant of fermentation broth

The enzymatic activity was monitored by change in absorbance at 610 nm for 10 minutes. The identity of the expressed the peroxidase was confirmed by mass-spectroscopic analysis of fragments from a tryptic in-gel digest.

All constructs resulted in expression of at least about 0.5 g/l of active soybean peroxidase.

### Example 2

### Cloning and Expression of Royal Palm Tree Peroxidase

The amino acid sequence of Royal palm tree peroxidase (SEQ ID NO:4) is publicly available (Uniprot D1MPT2), but there is no information about the native secretion signal. The amino acids encoded in secretion signal of the soybean peroxidase were therefore fused to the N-terminal of the mature amino acid sequence of the royal palm tree peroxidase. A DNA sequence was designed to encode this amino acid sequence using codon optimization, as described above, for expression in Aspergillus oryzae. A suitable restriction site was added at either end to ease cloning and the DNA was synthezised by a commercial provider.

The synthetic gene encoding the peroxidase was ligated into the multiple cloning site of plasmid pEN12516 as a BamHI-AfllI fragment to generate construct SEQ ID NO: 34 using standard technologies of molecular biology. This construct was used as template in a PCR reaction with Primer 3 and Primer 4 resulting in a fragment with approximate size 1029 bp. The PCR product contains restriction sites at either end which allows ligation of an AvrlI-AfllI fragment into existing plasmids to generate constructs SEQ ID NOs: 22, 24, 26, 28, 30 and 32. These constructs contain different secretion signal and prepro sequences known to work well in Aspergillus oryzae. All constructed plasmids were initially transformed into E. coli strain TOP10 and the inserts were sequenced to confirm nucleotide sequences. The plasmid was subsequently transformed into Aspergillus oryzae strain ToC1512 for expression trials.

The transformed strain of A. oryzae was grown for expression of peroxidase enzyme. Typically, 200 μL of YP growth medium was inoculated with spores from strains grown on sucrose agar added 10 mM NaNO₃. The cultures were grown in a 96 well sterile microplate for 3-4 days at 34° C. without shaking. Expression of peroxidase was confirmed by presence of a band with the correct molecular weight on SDS-PAGE and by activity on ABTS:
20 μL 10 mM ABTS
20 μL 0.3% hydrogen peroxidase
140 μL 100 mM Britton-Robinson buffer pH 3
10 μL Supernatant of fermentation broth

The enzymatic activity was monitored by change in absorbance at 405 nm for 5 minutes. The identity of the expressed the peroxidase was confirmed by mass-spectroscopic analysis of fragments from a tryptic in-gel digest.

All constructs resulted in expression of at least about 0.5 g/l of active royal palm tree peroxidase.

### Example 3

### Cloning and Expression of Poplar Peroxidase

A DNA sequence was designed to encode the amino acid sequence of poplar peroxidase (mature peroxidise is amino acids 38 to 354 of SEQ ID NO: 45) using codon optimization as described above. The gene was specifically designed for expression in Aspergillus oryzae and a restriction site was added at either end to ease cloning. The DNA was subsequently synthezised by a commercial provider.

The synthetic gene encoding the peroxidase was ligated into the multiple cloning site of plasmid pEN12516 as a BamHI-AfllI fragment to generate construct SEQ ID NO: 44 using standard technologies of molecular biology. This construct was used as template in a PCR reaction with Primer 5 and Primer 6 resulting in a fragment with approximate size 977 bp. The PCR product contains restriction sites at either end which allows ligation of an AvrlI-AfllI fragment into existing plasmids to generate constructs SEQ ID NOs: 46, 48, 50, and 52. These constructs contain different secretion signal and prepro sequences known to work well in Aspergillus oryzae. All constructed plasmids were initially transformed into E. coli strain Top10 and the inserts were sequenced to confirm nucleotide sequences. The plasmid was subsequently transformed into Aspergillus oryzae strain ToC1512 for expression trials.

The transformed strain of A. oryzae was grown for expression of peroxidase enzyme. Typically, 200 μL of YP growth medium was inoculated with spores from strains grown on sucrose agar added 10 mM NaNO₃. The cultures were grown in a 96 well sterile microplate for 3-4 days at 34° C. without shaking. Expression of peroxidase was confirmed by presence of a band with the correct molecular weight on SDS-PAGE and by activity on ABTS:
20 μL 10 mM ABTS
20 μL 0.3% hydrogen peroxide
140 μL 100 mM Britton-Robinson buffer pH 3
10 μL Supernatant of fermentation broth

The enzymatic activity was monitored by change in absorbance at 405 nm for 5 minutes. All constructs resulted in expression of at least about 0.5 g/l of active poplar peroxidase.

### Example 4

### Cloning and Expression of Maize Peroxidase

A DNA sequence was designed to encode the amino acid sequence of maize peroxidase (mature peroxidase is amino acids 30 to 362 of SEQ ID NO: 55) using codon optimization as described above. The gene was specifically designed for expression in Aspergillus oryzae and a restriction site was added at either end to ease cloning. The DNA was subsequently synthezised by a commercial provider.

The synthetic gene encoding the peroxidase was ligated into the multiple cloning site of plasmid pEN12516 as a BamHI-AfllI fragment to generate construct SEQ ID NO: 54 using standard technologies of molecular biology. This construct was used as template in a PCR reaction with Primer 7 and Primer 8 resulting in a fragment with approximate size 1023 bp. The PCR product contains restriction sites at either end which allows ligation of an AvrlI-AfllI fragment into existing plasmids to generate constructs SEQ ID NOs: 56, 58, 60, 62, and 64. These constructs contain different secretion signal and prepro sequences known to work well in Aspergillus oryzae. All constructed plasmids were initially transformed into E. coli strain Top10 and the inserts were sequenced to confirm nucleotide sequences. The plasmid was subsequently transformed into Aspergillus oryzae strain ToC1512 for expression trials.

The transformed strain of A. oryzae was grown for expression of peroxidase enzyme. Typically, 200 μL of YP growth medium was inoculated with spores from strains grown on sucrose agar added 10 mM NaNO₃. The cultures were grown in a 96 well sterile microplate for 3-4 days at 34° C. without shaking. Expression of peroxidase was confirmed by presence of a band with the correct molecular weight on SDS-PAGE and by activity on ABTS:
20 μL 10 mM ABTS
20 μL 0.3% hydrogen peroxidase
140 μL 100 mM Britton-Robinson buffer pH 3
10 μL Supernatant of fermentation broth

The enzymatic activity was monitored by change in absorbance at 405 nm for 5 minutes. All constructs resulted in expression of at least about 0.5 g/I of active maize peroxidase.

### Example 5

### Cloning and Expression of Tobacco Peroxidase

A DNA sequence was designed to encode the amino acid sequence of tobacco peroxidase (mature peroxidase is amino acids 23 to 324 of SEQ ID NO: 67) using codon optimization as described above. The gene was specifically designed for expression in Aspergillus oryzae and a restriction site was added at either end to ease cloning. The DNA was subsequently synthezised by a commercial provider.

The synthetic gene encoding the peroxidase was ligated into the multiple cloning site of plasmid pEN12516 as a BamHI-AfllI fragment to generate construct SEQ ID NO: 66 using standard technologies of molecular biology. The constructed plasmid was initially transformed into E. coli strain Top10 and the insert was sequenced to confirm nucleotide sequence. The plasmid was subsequently transformed into Aspergillus oryzae strain ToC1512 for expression trials.

The transformed strain of A. oryzae was grown for expression of peroxidase enzyme. Typically, 200 μL of YP growth medium was inoculated with spores from the strain grown on sucrose agar added 10 mM NaNO₃. The cultures were grown in a 96 well sterile microplate for 3-4 days at 34° C. without shaking. Expression of peroxidase was confirmed by presence of a band with the correct molecular weight on SDS-PAGE and by activity on ABTS:
20 μL 10 mM ABTS
20 μL 0.3% hydrogen peroxide
140 μL 100 mM Britton-Robinson buffer pH 3
10 μL Supernatant of fermentation broth

The enzymatic activity was monitored by change in absorbance at 405 nm for 5 minutes. The construct resulted in expression of at least about 0.5 g/I of active tobacco peroxidase.

1-24. (canceled)

25. A method for recombinant expression of a plant peroxidase, comprising expressing in a filamentous fungal host organism a nucleic acid sequence encoding a peroxidase, wherein the amino acid sequence of the peroxidase comprises one, two or three amino acid motifs selected from the group consisting of:
HFHDCFV;
GCD[A, G]S[V, I][I, L][I, L];
and
VSC[A, S]D[I, L][I, L].

26. The method of claim 25, wherein the motifs are selected from the group consisting of:
HFHDCFV;
GCD[A, G]S[V, I]LL;
and
VSC[A, S]D[I, L]L.

27. The method of claim 25, wherein the peroxidase is a class III peroxidase from EC 1.11.1.7

28. The method of claim 25, wherein the amino acid sequence of the peroxidase has at least 65% identity to the amino acid sequence of SEQ ID NO:2, SEQ ID NO:4, amino acids 38 to 354 of SEQ ID NO: 45, amino acids 30 to 362 of SEQ ID NO: 55, or amino acids 23 to 324 of SEQ ID NO: 67.

29. The method of claim 25, wherein the peroxidase consists of the amino acid sequence of SEQ ID NO:2, SEQ ID NO:4, amino acids 38 to 354 of SEQ ID NO: 45, amino acids 30 to 362 of SEQ ID NO: 55, or amino acids 23 to 324 of SEQ ID NO: 67.

30. The method of claim 25, wherein the nucleic acid sequence is attached to suitable control sequence(s) that provide for expression of the peroxidase.

31. The method of claim 25, wherein at least one codon of the nucleic acid sequence is optimized for translation in a filamentous fungal host organism.

32. The method of claim 25, wherein at least half of the codons of the nucleic acid sequence are optimized for translation in a filamentous fungal host organism.

33. The method of claim 25, wherein the nucleic acid sequence is codon optimized in at least 10% of the codons.

34. The method of claim 31, wherein the optimized codon(s) corresponds to the codon usage of alpha amylase from Aspergillus oryzae.

35. The method of claim 25, wherein the filamentous fungal host organism is selected from the group consisting of Acremonium, Aspergillus, Fusarium, Humicola, Mucor, Myceliophthora, Neurospora, Penicillium, Thielavia, Tolypocladium, or Trichoderma.

36. The method of claim 25, wherein the filamentous fungal host organism is an Aspergillus sp., preferably Aspergillus awamori, Aspergillus foetidus, Aspergillus japonicus, Aspergillus niger, Aspergillus nidulans, or Aspergillus oryzae.

37. A modified nucleic acid sequence encoding a wild type peroxidase and capable of expression in a filamentous fungal host organism, wherein said modified nucleic acid sequence differs in at least one codon from the wild type nucleic acid sequence encoding the wild type peroxidase, and wherein the peroxidase has at least 60% identity to soy bean peroxidase or royal palm tree peroxidase and comprises one, two or three amino acid motifs selected from the group consisting of:
HFHDCFV;
GCD[A, G]S[V, I]LL;
and
VSC[A, S]D[I, L]L.

38. The modified nucleic acid sequence of claim 37, wherein the modification of at least one codon is optimized for translation in an Aspergillus host organism.

39. The modified nucleic acid sequence of claim 38, wherein the Aspergillus host organism is Aspergillus awamori, Aspergillus foetidus, Aspergillus japonicus, Aspergillus niger, Aspergillus nidulans, or Aspergillus oryzae.

40. The modified nucleic acid sequence of claim 37, wherein the codon usage corresponds to the codon usage of alpha amylase from Aspergillus oryzae.

41. The modified nucleic acid sequence of claim 37, which is shown as SEQ ID NO: 1, 3, 8, 10, 12, 14, 16, 18, 20, 22, 24, 26, 28, 30, 32, 34, 44, 46, 48, 50, 52, 54, 56, 58, 60, 62, 64, or 66.

42. A modified nucleic acid sequence encoding a peroxidase and capable of expression in a filamentous fungal host organism, which has at least 50% identity to a nucleic acid sequence selected from the group consisting of SEQ ID NOs: 1, 3, 8, 10, 12, 14, 16, 18, 20, 22, 24, 26, 28, 30, 32, 34, 44, 46, 48, 50, 52, 54, 56, 58, 60, 62, 64, and 66.

43. A recombinant filamentous fungal host organism, comprising the modified nucleic acid sequence of claim 37.

44. The recombinant filamentous fungal host organism of claim 43, which is an Aspergillus sp.
